(19) United States
(12) Patent Application Publication
(10) Pub. No.: US 2019/0392920 A1
(43) Pub. Date: Dec. 26, 2019

(54) METHOD OF VALIDATING MRNA SPLICING MUTATIONS IN COMPLETE TRANSCRIPTOMES

(71) Applicant: Cytognomix Inc, London (CA)

(72) Inventors: Peter Keith Rogan, London (CA); Stephanie Nicole Dorman, London (CA); Coby Viner, London (CA); Eliseos John Mucaki, London (CA)

(21) Appl. No.: 16/448,064

(22) Filed: Jun. 21, 2019

Related U.S. Application Data
(63) Continuation of application No. 14/594,109, filed on Jan. 10, 2015, now abandoned.

(51) Int. Cl. G16B 20/00; G16C 20/60; G16B 35/00; C12Q 1/6883

(52) U.S. Cl. CPC G16B 20/00; G16C 20/60; G16B 35/00; C12Q 2600/16; C12Q 2600/118; C12Q 2600/156; C12Q 1/6883

(57) ABSTRACT

A method is described for the automatic validation of DNA sequencing variants that alter mRNA splicing from nucleic acids isolated from a patient or tissue sample. Evidence of a predicted splicing mutation is demonstrated by performing statistically valid comparisons between sequence read counts of abnormal RNA species in mutant versus non-mutant tissues. The method leverages large numbers of control samples to corroborate the consequences of predicted splicing variants in complete genomes and exomes for individuals carrying such mutations. Because the method examines all transcript evidence in a genome, it is not necessary a priori to know which gene or genes carry a splicing mutation.

### RELATED APPLICATIONS

This continuation-in-part application claims priority from U.S. Non-Provisional application Ser. No. 14/594,109 filed on Jan. 10, 2015, which in turn claims priority from U.S. Provisional Applications Nos. 61/926,312 and 62/044,403, respectively filed on Jan. 11, 2014 and Sep. 1, 2014 and, the content of which is hereby incorporated into this application by reference.

### BACKGROUND OF THE INVENTION

### I. Field of the Invention

The present method relates to experimental validation of in silico predicted cryptic, exon skipping and unspliced isoforms in mRNA produced by splicing mutations. The method allows for streamlining assessment of abnormal and normal splice isoforms resulting from such mutations in patients with genetic diseases and other phenotypes.

### II. Description of the Related Art

mRNA processing mutations, which are responsible for a wide range of human diseases (Divina et al., 2009), alter the abundance and/or structures of mature transcripts. This type of mutation has been hypothesized to be the most frequent cause of hereditary disease (López-Bigas et al., 2005). These mutations often occur proximate to exon/intron boundaries, but are frequently found at other sequence locations within introns or exons. Mutations which abolish or weaken recognition of natural splice acceptor or donor sites often produce transcripts lacking corresponding exons or activate adjacent cryptic splice sites of the same phase. Alternatively, mutations activate cryptic splice sites whose strength exceeds existing natural sites elsewhere in the unspliced transcript. The resultant molecular phenotypes may include isoforms with altered exon length and, in some instances, reduced or leaky expression of normal isoforms. The instant invention is an approach to validate predicted structures and approximate abundance of the output molecules generated directly or indirectly by splicing mutations.

Berget's exon definition model (Berget, 1995) provides a mechanism for recognizing multiple small exons against a background of considerably larger intronic sequences. Accurate exon recognition can be complicated by pseudo-exonic structures present in introns that mimic natural exon structures (Ibrahim et al., 2005). To discriminate between these structures, accurate spliceosomal recognition relies on relatively high affinities of the recognition sequences in natural exons and the presence of other splicing regulatory elements. Exons and adjacent introns also contain splicing enhancer (ESE, ISE) and silencer (ESS, ISS) sequences close to or overlapping constitutive splice sites, which may assist or suppress exon recognition through interactions with additional proteins (Berget, 1995; Graveley and Maniatis, 1998). Recognition of an exon may therefore depend to some degree on the combined effects of each of these proteins (Goren et al., 2010), however the factors that recognize the acceptor and donor splice sites are often sufficient (Hwang and Cohen, 1997).

Information theory can be used to measure the conservation of nucleotide sequences bound by individual proteins or protein complexes. In splicing, information theory-based models of donor and acceptor splice sites reveal which nucleotides are permissible at both highly conserved and variable positions in individual sites (Schneider, 1997; Robberson et al., 1990; U.S. Pat. No. 5,867,402). These sequences are recognized prior to intron excision, these recognition events are concerted, and related to the binding strength of the spliceosome-splice site interaction (Berget, 1995). The strengths of spliceosome-splice site interactions are related to the corresponding individual information content, Rᵢ, of the RNA sequence (Rogan et al., 1998; Caminsky et al. 2014). As disclosed here, an exon may be defined by the cumulative Rᵢ values of each of these distinct binding sites contributing to exon recognition (R_{i,total}), based on the fact that information is additive for independent sources of uncertainty (Jaynes 1957).

Computational identification of mRNA splicing mutations within DNA sequencing (DNA-Seq) data has been implemented to varying degrees of sensitivity, with most software only evaluating conservation solely at the intronic dinucleotides adjacent to the junction (i.e. Wang et al. 2010). Other approaches are capable of detecting significant mutations at other positions with constitutive, and in certain instances, cryptic, splice sites [5, 8, 9] which can result in aberrations in mRNA splicing. Previously described bioinformatic methods that predict the effects of mutations that could alter mRNA splicing generally examine the effect of a single gene variant in situ, at or proximate to the mutation itself. Among these programs are Cryp-SKIP, SpliceScan II (Churbanov et al. 2010), Annovar pipeline, Bayesian sensor (Churbanov et al. 2006) and SpliceScan tool (Churbanov et al. 2006), Alamut software a commercial product that includes (implementation of the published SSF-like, Max-EntScan, NNSPlice, and GeneSplicer algorithms). Alamut software has been used in a recent study of aberrant splicing prediction (Thomassen et al. 2012) and has been found to be sensitive, but not specific (Spurdle et al. 2012). None of these prior art computations not make reference to, incorporate, or anticipate exon recognition processes. While machine learning methods have been developed to predict alternatively spliced transcripts, a natural process that occurs in cells with a normal genotype (Barash et al, 2010), these ad hoc methods are not supported by a rigorous theoretical framework that relates the predicted isoforms to thermodynamic binding affinity and thus cannot be used to analysis of the relative abundance of different isoforms. CRYP-SKIP is another bioinformatic method which employs multiple logistic regression to predict the two aberrant transcripts from the primary sequence (Divina et al., 2009). It predicts the overall probability of cryptic splice-site activation as opposed to exon skipping, which has some resemblance to exon definition. However, the online resource developed for this method does not take into consideration the impact of mutations. Although a user can simply analyze the wildtype and mutated sequences individually and compare them manually, such method is not based on information theory, nor does it use the gap surprisal function to factor exon size penalties.

DNA variant analysis of complete genome or exome data has typically relied on filtering of alleles according to population frequency and alterations in coding of amino acids. Numerous variants of unknown significance (VUS) in both coding and non-coding gene regions cannot be categorized with these approaches. To address these limitations, in silico methods that predict biological impact of individual sequence variants on protein coding and gene expression have been developed, which exhibit varying degrees of sensitivity and specificity (Rogan and Zou 2013). These approaches have generally not been capable of objective, efficient variant analysis on a genome-scale.

Presently, only information theory-based mRNA splicing mutation analysis has been implemented on a genome scale (Shirley et al. 2013; U.S. Pat. No. 5,867,401). Splicing mutations can abrogate recognition of natural, constitutive splice sites (inactivating mutation), weaken their binding affinity (leaky mutation), or alter splicing regulatory protein binding sites that participate in exon definition. The abnormal molecular phenotypes of these mutations comprise: (a) complete exon skipping, (b) reduced efficiency of splicing, (c) failure to remove introns (also termed intron retention or intron inclusion), or (d) cryptic splice site activation, which may define abnormal exon boundaries in transcripts using non-constitutive, proximate sequences, extending or truncating the exon. Some mutations may result in combinations of these molecular phenotypes. Nevertheless, novel or strengthened cryptic sites can be activated independently of any direct effect on the corresponding natural splice site. The prevalence of these splicing events has been determined by ourselves and others (Mucaki et al. 2013, Eswaran et al. 2012, Eswaran et al. 2013, Kwan et al. 2008). The diversity of possible molecular phenotypes makes such aberrant splicing challenging to corroborate at the scale required for complete genome (or exome) analyses. This has motivated the development of statistically robust algorithms and software to comprehensively validate the predicted outcomes of splicing mutation analysis.

Putative splicing variants require empirical confirmation based on expression studies from appropriate tissues carrying the mutation, compared with control samples lacking the mutation. In mutations identified from complete genome or exome sequences, corresponding transcriptome analysis based on RNA sequencing (RNA-Seq) is performed to corroborate variants predicted to alter splicing. Manually inspecting a large set of splicing variants of interest with reference to the experimental samples' RNA-Seq data in a program like the Integrative Genomics Viewer (IGV; Thorvaldsdóttir et. 2013), or simply performing database searches to find existing evidence for splicing abberations is time-consuming and impractical for large-scale analyses of, for example, multiple genomes. Checking control samples would be required to ensure that the variant is not a result of alternative splicing, but is actually causally linked to the variant of interest. Manual inspection of the number of control samples required for statistical power to verify that each displays normal splicing would be laborious and does not easily lend itself to statistical analyses. This may lead to either missing contradictory evidence or to discarding a variant due to the perceived observation of statistically insignificant altered splicing within control samples. In addition, a list of putative splicing variants returned by variant prediction software can often be extremely large. The validation of such a significant quantity of variants may not be feasible, for example, in certain types of cancer, in instances where the genomic mutational load is high and only manual annotation is performed. We have therefore developed the instant invention, termed Veridical, a method and a software program that automatically searches all given experimental and control RNA-Seq data to validate DNA-derived splicing variants. When adequate expression data are available at the locus carrying the mutation, this approach reveals a comprehensive set of genes exhibiting mRNA splicing defects in complete genomes, exomes and/or panels of gene sequences.

### SUMMARY

In contrast with splice sites across an intron, cognate pairs of donor and acceptor splice sites from the same exon tend to be separated by a narrow range of distances in the unspliced transcript. Single exon recognition tends to be constrained by preferred distances between the U2 and U1 spliceosomal binding sites across the same exon (Hwang and Cohen, 1997). A model to define exon sequences that incorporates the information contents of both splice sites and preferences for certain exon lengths of all natural exons has been previously presented (Rogan, 2009). A general approach is used that minimized entropy of a pair of binding sites separated by a variable length interstitial sequence. Given a set of exons flanked on either side by 100 nucleotides (nt) intron sequences, the most accurate model (99% correctly detected exon boundaries) was derived by bootstrapping sets of 4000 sequences with left (acceptor) and right (donor) sites of 31 (9.7 bits) and 15 nts (8.1 bits) in length. Efforts are used to ensure that pairs of splice sites of opposite polarity are derived from the same exon by incorporating the surprisal function (Tribus, 1961), also termed self-information by Shannon (Cover and Thomas, 2006), which corrects for both frequent and uncommon or rare inter-site distances that are unlikely to form an exon. This is based on the observation that long internal exons are recognized inefficiently (Robberson et al., 1990), though they do occur (1115 known internal exons >1000 nt; (Bolisetty and Beemon, 2012). The total exon information content (R_{i,total}) is significantly reduced by this gap surprisal value, if either the predicted exon length is suboptimal or splice site pairs are derived from different exons, but is nearly unchanged for common exon lengths. Computation of R_{i,total} and the use of this value for predicting natural and mutated splice isoforms and relative abundance of these isoforms with respect to one another due to mutations in the genome are described in more detail in U.S. patent application Ser. No. 14/154,905), which is hereby incorporated by reference.

The present disclosure provides a novel and previously unknown method for validation of the effect of a predicted splicing mutation on the relative abundance of natural and cryptic splice isoforms using the exon definition model. The method may contain, among others, the following steps:

(a) Isolation of DNA and RNA from one or more tissue or blood samples (including cell cultures) using standard molecular biological and biochemical methods;
(b) Determining the genomic DNA sequence in one or more samples for either a single or multiple genes, or an exome or a complete genome using standard Sanger sequencing methods or massively parallel sequencing methods in common use;
(c) Preparing cDNA from total RNA from one or more samples
(d) Sequencing of the transcriptome(s) of the samples, which is typically done by massively parallel sequencing methods in common use (and is termed RNASeq);
(e) Predicting that a sequence contains a splicing mutation by calculating the information content of all donor (eg. 5′) and acceptor (eg. 3′) splice sites within a given genomic region, in the normal reference sequence (eg. before the mutation) and the mutant sequence (eg. after the mutation) and/or by calculating the total information content of every potential exon before and after mutation, and ranking them in descending order post-mutation;
(f) Experimentally validating of predicted splicing mutations from a sample which result in intron inclusion, i.e., the failure to excise intronic sequences between two exons, with the sequence data from RNASeq analysis of the same sample by performing the following steps:
A) extracting and reverse transcribing mRNA from a cell from a patient with the disease, and characterizing the isoforms of each expressed, mutated gene by:
   i) counting the number sequenced RNA templates in a sequence library containing at least one intronic nucleotide in a sample, the ζᵢ, evidence for intron inclusion in the patient sample that contains a mutation in the corresponding genomic sequence of either the same intron or the adjacent proximate exon, said mutation having been first predicted to alter the structure of the mRNA transcript, and
   ii) counting ζᵢ, evidence for intron inclusion in control samples, from the number of sequence reads derived from RNA templates containing at least one intronic nucleotide in one or more control samples that do not contain the same predicted splicing mutation in the corresponding genomic sequence, and
   iii) determining the probability that the mutation alters the mRNA structure of a gene from the count of sequence reads in the sample containing the predicted mutation computed in step (i) and the number of counts of sequence reads in the set of control samples computed in step (ii), as:

$$μ = \frac{{\sum }_{j = 1}^{N} {V}_{j}}{N} σ = \sqrt{\frac{1}{N} {\sum }_{j = 1}^{N} {( {V}_{j} - \overline{V} )}^{2}}$$

$$z = \frac{\vert {Ϛ}_{i} \vert - μ}{σ} p = Φ ( ψ ( z , \frac{1}{2} ) )$$

where ϕ_{z} (z) represents the cumulative distribution function of read counts of the one-sided (right-tailed, i.e., P[X>x]) standard normal distribution with mean μ and standard deviation σ, z is the distance from μ for ζᵢ reads, N represents the total number of samples and V represents the set of all ζᵢvalidations, across all samples.
B) validating that a predicted mutation is an actual mutation, if the probability of sequence read evidence present in the disease carrier is less than or equal to 0.05499.
   (g) Experimentally validating of predicted splicing mutations from a sample which result in exon skipping, i.e. derived from non-consecutive exons from the same gene, with the sequence data from RNASeq analysis of the same sample by performing the following steps:
A) extracting and reverse transcribing mRNA from a cell from a patient with the disease, and characterizing the isoforms of each expressed, mutated gene by:
   i) counting the number sequenced RNA templates in a sequence library containing at least one abnormal splice junction derived from non-consecutive exons from the same gene in a sample i.e. exon skipping, ζₑ, the evidence for exon skipping in the patient sample that contains a mutation in the corresponding genomic sequence adjacent to the splice junction of a proximate exon, said mutation having been first predicted to alter the structure of the mRNA transcript, and
   ii) counting ζₑ, evidence for exon skipping in control samples, from the number of sequence reads derived from RNA templates containing the same abnormal splice junction present in the patient sample in one or more control samples that do not contain the same predicted splicing mutation in the control genomic sequences, and
   iii) determining the probability, P, that the mutation alters the mRNA structure of a gene from the count of sequence reads in the sample containing the predicted mutation computed in step (i) and the number of counts of sequence reads in the set of control samples computed in step (ii), as:

$$μ = \frac{{\sum }_{j = 1}^{N} {V}_{j}}{N} σ = \sqrt{\frac{1}{N} {\sum }_{j = 1}^{N} {( {V}_{j} - \overline{V} )}^{2}}$$

$$z = \frac{\vert {Ϛ}_{i} \vert - μ}{σ} p = Φ ( ψ ( z , \frac{1}{2} ) )$$

where ϕ_{z} (z) represents the cumulative distribution function of read counts of the one-sided (right-tailed, i.e., P[X>x]) of the standard normal distribution with mean μ and standard deviation σ, z is the distance from μ for ζₑ reads, N is the total number of samples and V represents the set of all ζₑ validations, across all samples.
B) validating that a predicted mutation is an actual mutation, if the probability of sequence read evidence present in the disease carrier is less than or equal to 0.05499.

It is an object of the present disclosure to use information-theory based exon definition models to test predictions of splice isoforms activated and deactivated by splicing mutations, which can reveal and confirm splice isoforms that have not been previously described.

It is an object of the present disclosure to be able to predict and experimentally validate the relative abundance of these wild-type and mutated splice forms comparison of total exon information or changes in individual information values.

It is an object of the present disclosure to factor and experimentally validate splicing mutation-directed changes in splicing enhancers and silencers (small nuclear ribonucleoproteins; snRNPs) into the total exon information calculation. A second snRNP-specific gap surprisal function, which is based on the common distance between a natural splice site and the nearest predicted splicing enhancer of the same type, would also be applied.

It is disclosed here a novel approach to predict and experimentally validate the molecular phenotype of a splicing mutation, producing a probable set of splicing isoforms expressed in mutation carriers. The system is based on information theory-based methods that accurately quantify binding site affinity (Schneider, 1997; Rogan et al., 1998). Non-expressed or very low expression exons are filtered out by correcting for suboptimal exon lengths and eliminating incorrectly ordered splice sites.

It is also shown here a simple model for exon definition based on constitutive splice sites, although the theory for extensible framework for incorporation of multiple splice site recognition sequences is derived and validated with experimental data. Exon definition-based predictions were compared to known splicing mutations with published mRNA studies, and these predictions were found to be highly concordant (see FIG. 8A, FIG. 8B, FIG. 8C, FIG. 8D, and FIG. 8E). These mutations were sourced from our previous publications so that information theory-based modelling of individual splice sites could be compared with exon definition (Rogan et al., 1998; Mucaki et al., 2011).

Information analysis correctly predicted several types of splicing abnormalities in different genes (Mucaki et al., 2013). The development of exon definition-based mutation analysis was motivated by the desire to generate predictions that could be directly compared with laboratory expression data. In some instances, these predictions have included strong cryptic exons that have not been previously detected, possibly because the laboratory studies did not directly anticipate the corresponding splice isoforms. The level of concordance we report for previously validated splicing mutations justifies a prospective study of natural and mutant isoforms predicted by the server, in which all predicted cryptic splice isoforms (including exon skipped isoforms) are tested, and if possible, quantified. The instant invention provides a method for objectively quantifying these isoforms. It has the advantage of closing the circle between bioinformatic methods that predict potential splicing mutations in large scale genomic DNA sequence studies and validation with mRNA obtained from the same individuals.

In one embodiment, it is disclosed here a method for assessing and verifying changes in expression level of a gene of interest. In one aspect, the gene has an mRNA splice-altering mutation. In another aspect, the mutation is located within a sequence window circumscribing an exon and one or more intronic sequences of the gene, where the one or more intronic sequences are adjacent to the exon.

In another embodiment, the mutation may occur at a cryptic splice site. For instance, the mutation may be a leaky or partial splicing mutation, which causes a mutant isoform to exceed the abundance of the normal mRNA splice isoform by at least 1 bit or 2 fold. In one aspect, the mutation may result from a paucimorphic allele or an effectively null allele in which a mutant isoform exceeds the abundance of the normal mRNA splice isoform by at least 5 bits, which is equivalent to a 32 fold change.

In another embodiment, the mutation may occur at a natural splice site. For example, the mutation may be a leaky or partial splicing mutation, which causes the R_{i,total} of the mutant isoform to be less than the R_{i,total} value of the normal mRNA splice isoform by at least 1 bit or 2 fold. In one aspect, the mutation may result from a paucimorphic or an effectively null allele in which the R_{i,total} of the mutant isoform is less than the R_{i,total} value of the normal mRNA splice isoform by at least 5 bits which is equivalent to a 32 fold change.

The method may include at least the following steps (a)-(d): (a) computing and identifying changes in the individual information contents of potential donor and acceptor splice sites at each nucleotide position by computing product of the information theory-based position weight matrices and a unitary position matrix of each sequence; (b) defining potential exons by selecting every pair combination of acceptor and donor splice sites in the sequence window, and determining the gap surprisal value based on distance in nucleotides between sites comprising a pair combination, wherein, the gap surprisal value is calculated for each potential exon length based on frequency of said length in the genome as the inverse log₂ of said frequency; (c) computing the total information content, R_{i,total}, of a potential exon as the sum of the corresponding individual information contents of the acceptor and donor pair, corrected by adding the gap surprisal of an exon whose length is the distance between the donor and acceptor pair; and (d) comparing the R_{i,total} values of all potential mRNA splice isoforms of the wild-type gene and the same vales after the wild-type gene sequence is mutated to determine whether the mutation alters the abundance of the mRNA isoforms containing the exon, wherein the splice isoform with the largest R_{i,total} value is predicted to be the most abundant splice isoform, and the splice isoform with the smallest R_{i,total} value is the least abundant isoform.

In another embodiment, the method may also include a step of extracting mRNAs or proteins from at least one cell expressing the gene to determine the most abundant mRNA splice isoform of the gene, thus allowing the assessing of changes in expression level of the gene. In one aspect, the extracting step may be performed by extracting mRNAs from said at least one cell and by determining the sequence of one or more mRNA molecules derived from the gene. In another aspect, the extracting step is performed by extracting proteins from said at least one cell expressing said gene and by determining the sequence of one or more protein molecules derived from the gene of interest.

In another embodiment, the method may also include a step of introducing the gene into at least one cell and extracting mRNAs or proteins from the at least one cell expressing the gene to determine the most abundant mRNA splice isoform of the gene, thus allowing the assessing of changes in expression level of the gene.

In another embodiment, the steps (a)-(d) above are preceded by a step of generating a genomic polynucleotide sequence of the gene of interest. In one aspect, the genomic polynucleotide sequence may be generated by isolating genomic DNA from a cell containing the gene and by sequencing the isolated genomic DNA using PCR, conventional sequencing or other sequencing techniques, such as mass spectrometry.

In another embodiment, the comparison step (d) may be performed by determining the relative abundance of a pair of splice isoforms by computing 2 to the power of the difference between the R_{i,total} values of each isoform.

In one aspect, the disclosed method may be specific for first exons, using a first exon-specific gap surprisal function. In another aspect, the disclosed method may be specific for last exons, using a last exon-specific gap surprisal function.

In another embodiment, the method adds a component that takes into account one or more splicing enhancer or silencer sequence elements recognized by RNA binding proteins or small nuclear ribonucleoproteins, wherein strength of at least one of the splicing enhancer or silencer sequence elements is altered due to the mutation.

In another embodiment, a secondary gap surprisal may be applied to take into account distances between the natural site and each of the altered splicing enhancer or silencer sequence elements.

### Advantages of the Method

The Veridical method automates confirmation of mRNA splicing mutations by comparing sequence read-mapped expression data from samples containing variants that are predicted to cause defective splicing with control samples lacking these mutations. The program objectively evaluates each mutation with statistical tests that determine the likelihood of and exclude normal splicing. When Veridical was first implemented, no other method was available to automatically validate splicing mutations with RNA-Seq transcriptome data on a transcriptome-wide scale, although many applications have been described that accurately detect conventional alternative splice isoforms (for example, Shen et al. 2012). Veridical is intended for use with large data sets derived from many samples, each containing several hundred variants that have been previously prioritized as likely splicing mutations, regardless of how the candidate mutations are selected. It is not practical to computationally to analyze all variants present in an exome or genome, rather only a filtered subset, due to the extensive computations required for statistical validation. Veridical is a key component of an end-to-end, hypothesis-based, splicing mutation analysis framework that we have implemented (Mucaki et al. 2013; Shirley et al. 2013). There is a trade-off between lengthy run-times and statistical robustness of Veridical, especially when there are either a large number of variants or a large number of RNA-Seq files. As with most statistical methods, those employed here are not amenable to small sample sets, but become quite powerful when a large number of controls are employed. In order to ensure that mutations can be validated, we recommend an excess of control transcriptome data relative to those from samples containing mutations (>5:1), guided by the power analysis described herein. Use of a single nor a few control samples to corroborate a putative mutation is not recommended. Junction-spanning reads have the greatest value for corroborating cryptic splicing and exon skipping. Even a single such read is almost always sufficient to merit the validation of a variant, provided that sufficient control samples are used. For intron inclusion, both junction-spanning and read-abundance-based reads are useful and a variant can readily be validated with either, provided that the variant-containing experimental sample(s) show a statistically significant increase in the presence of either form of intron inclusion corroborating reads.

Veridical is able to automatically process variants from multiple different experimental samples, and can group the variant information if any given mutation is present in more than one sample. The use of a large sample size allows for robust statistical analyses to be performed, which aid significantly in the interpretation of results. The main utility of Veridical is to filter through large data sets of predicted splicing mutations to prioritize the variants. This helps to predict which variants will have a deleterious effect upon the protein product. Veridical is able to avoid reporting splicing changes that are naturally occurring through checking all variant-containing and non-containing control samples for the predicted splicing consequence. In addition, running multiple samples at once allows for manual inspection to discover samples that contained the alternative splicing pattern, and consequently, permits the identification of DNA mutations in the same location which went undetected during genome sequencing.

The statistical power of Veridical is dependent upon the quality of the RNA-Seq data used to validate putative variants. In particular, a lack of sufficient coverage at a particular locus will cause Veridical to be unable to report any significant results. A coverage of at least 20 reads should be sufficient. This estimate is based upon alternative splicing analyses in which this threshold was found to imply concordance with microarray and RT-PCR measurements (Griffith et al. 2010; Katz et al. 2010; Shen et al. 2011; Kapranov et al. 2007; Feng et al. 2013). There are many potential legitimate reasons why a mutation may not be validated: (a) A lack of gene expression in the variant containing tumour sample, (b) nonsense-mediated decay may result in a loss of expression of the entire transcript, (c) the gene itself may have multiple paralogs and reads may not be unambiguously mapped, (d) other non-splicing mutations could account for a loss of expression, and (e) confounding natural alternative splicing isoforms may result in a loss of statistical significance during read mapping of the control samples. The prevalence of loci with insufficient data is dependent upon the coverage of the sequencing technology used. As sequencing technologies improve, the proportion of validated mutations is expected to increase. Such an increase would mirror that observed for the prevalence of alternative splicing events (Eswaran et al 2013). In addition, mutated splicing factors can disrupt splicing fidelity and exon definition (Pai et al. 2012). This effect could decrease Veridical's ability to validate splicing mutations affected by a disruption of the definition of the pertinent exon. Veridical does not currently form any equivalence between distinct variants affecting the same splice site. Such variants will be analyzed independently. Veridical is intended to be used with RNA-Seq data that not only corresponds to matched DNA-Seq data, but also only for sets of samples with comparable sequencing protocols, since the non-normalized comparisons performed rely upon the evening out of batch effects, due to a substantial number of control samples. It is important to note that acceptance of the null hypothesis, due to an absence of evidence required to disprove it, does not imply that the underlying prediction of a mutation at a particular locus is incorrect, but merely that the current empirical methods employed were insufficient to corroborate it. “Validate,” in the present context, refers to the condition where sufficient statistical evidence has been marshaled in support of a variant. However, the threshold for significance can vary so these analyses can also be thought of as strongly corroborating variants. Recent studies in Bayesian statistics have suggested that a p-value threshold of 0.05 does not correspond to strong support of the alternative hypothesis. Accordingly, Johnson (2013) recommends the use of tests at the 0.005 or 0.001 level of significance.

We consider alternative splicing to be a different problem. Veridical does not aim to identify putatively pathogenic variants, but rather, to confirm existing in silico predictions thereof. We do infer exon skipping events (i.e. alternative splicing) de novo, but only to catalog dysregulated splicing “phenotypes” due to genomic sequence variants. This is not the first study to use a large control dataset. Indeed the Variant Annotation, Analysis & Search Tool (VAAST; Yandell et al. 2011) does this to search for disease-causing (non-splicing) variants and the Multivariate Analysis of Transcript Splicing (MATS; Shen et al. 2012) tool (among others) can be used for the discovery of alternative splicing events. However, in our case, in most instances the distribution of reads in a single sample is compared to the distributions of reads in the control set, as opposed to a likelihood framework-based approach. We are suggesting that our approach be coupled to existing approaches to act as an a posteriori, hypothesis-driven, check on the veridicality of specific variants.

While there is considerable prior evidence for splicing mutations that alter natural and cryptic splice site recognition, we were somewhat surprised at the apparent high frequency of statistically significant intron inclusion revealed by Veridical. In fact, evidence indicates that a significant portion of the genome is transcribed (Kapranov et al. 2007), and it is estimated that 95% of known genes are alternatively spliced (Pan et al. 2008). Defective mRNA splicing can lead to multiple alternative transcripts including those with retained introns, cassette exons, alternate promoters/terminators, extended or truncated exons, and reduced exons (Feng et al 2013). In breast cancer, exon skipping and intron retention were observed to be the most common form of alternative splicing in triple negative, non-triple negative, and HER2 positive breast cancer (Eswaran et al. 2013). In normal tissue, intron retention and exon skipping has been predicted to affect 2572 exons in 2127 genes and 50 633 exons in 12 797 genes, respectively (Pai et al. 2012). In addition, previous studies suggest that the order of intron removal can influence the final mRNA transcript composition of exons and introns. Intron inclusion observed in normal tissue may result from those introns that are removed from the transcript at the end of mRNA splicing. Given that these splicing events are relatively common in normal tissues, it becomes all the more important to distinguish expression patterns that are clearly due to the effects of splicing mutations—one of the guiding principles of the Veridical method.

The instant invention is an important analytical resource for unsupervised, thorough validation of splicing mutations through the use of companion RNA-Seq data from the same samples. The approach will be broadly applicable for many types of genetic abnormalities, and should reveal numerous, previously unrecognized, mRNA splicing mutations in exome and complete genome sequences.

### BRIEF DESCRIPTION OF THE DRAWINGS

In order that the manner in which the above-recited and other advantages and objects of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1. Diagram portraying the definitions used within Veridical to specify genic variant position and read coordinates. FIG. 1 (A) shows the location of all reads extracted to analyze variant C. All reads overlapping or between D or E are extracted from the BAM files. We assume, for clarity of illustration, that the genome coordinate D<E. The variant, C, is contained somewhere within the middle exon or within one of its adjacent introns. FIG. 1 (B) shows a region searched for validating reads of the effect of variant C. Veridical searches for validating reads between A and B, the orientation of which is direction dependent. As indicated, the variant, C, is contained somewhere within the middle exon or within one of its adjacent introns. Depending upon the location of the variant, and the directionality (as described within Table 1), the interval boundaries may be delimited by either the blue (B-A) or red (A′-B′) set of labels.

FIG. 2. Illustrative examples of aberrant splicing detection. FIG. 2 (A) shows a normally spliced transcript for validation of splicing aberration of variant, indicated by C. An Example of a normally spliced transcript, assuming Veridical is validating a specific variant, C, shown in yellow. The adjacent intron-exon boundary, in this case, corresponds to both the adjacent splice junction, J_{c} and the relevant natural site A. B is the downstream natural site. Veridical would not identify any aberrant splicing. FIG. 2 (B) shows a variant, indicated by A, causing activation of a cryptic splice site. An example of the variant causing the activation of a cryptic splice site. Additionally, there is intron inclusion present within the analysis region. Veridical would identify and report read counts for reads pertaining to the (junction-spanning, purple) cryptic splicing event and those the pertaining to the observed (junction-spanning and read-abundance, green) intron inclusion. Since this pertains to a cryptic variant, the adjacent splice junction, J_{c} is distinct form the relevant natural site A. FIG. 2 (C) a variant (indicated by either D or E) causing the containing exon to be skipped. An example of the variant causing the containing exon to be skipped. Veridical would report read counts for reads pertaining to the junction-spanning exon skipping event. These discontinuous reads are those, that like the one shown, span the variant containing exon. Grey lines denote reads, wherein thick lines denote a read mapping to genomic sequence and thin lines represent connecting segments of reads split across spliced-in regions (i.e. exons or included introns). Dotted rectangles denote portions of genes which are spliced out in a mutant transcript, but are otherwise present in a normal transcript. Mutant reads are purple if they are junction-spanning and green if they are read-abundance based. Start and end coordinates of reads with two portions are denoted by

$$( {r}_{{s}_{1}} , {r}_{{e}_{1}} ) and ( {r}_{{s}_{2}} , {r}_{{e}_{2}} ) ,$$

while coordinates of those with only a single portion are denoted by (rₛ,rₑ). Refer to the caption of FIG. 1 for additional graphical element descriptions.

FIG. 3. The algorithm employed by Veridical to validate variants. Refer to Table 1 for definitions concerning direction and FIG. 1 for variable depictions. B is defined as follows: B (B site left (←) of A=⇒B:=D. B site right (→) of A⇒B:=E.

FIG. 4. IGV images depicting a predicted leaky mutation (chr5:162905690G>T) within the natural acceptor site of exon 12 (162905689-162905806) of HMMR. This gene has four transcript variants and the given exon number pertains to isoforms a and b (reference sequences NM_001142556 and NM_012484). RNA-Seq reads are shown in the centre panel. The bottom track depicts RefSeq genes, wherein each rectangle denotes an exon and connecting lines denote introns. In the middle panel, each rectangle (grey by default) denotes an aligned read, while thin lines are segments of reads split across exons. Rectangles in the middle panel denote aligned reads of inserts that are larger or smaller than expected, respectively. Reads are highlighted by their splicing consequence, as follows: cryptic splicing, exon skipping, junction-spanning intron inclusion, and read-abundance intron inclusion. (A) depicts a genomic region of chromosome 5: 162902054-162909787. The variant occurs in the middle exon. Intron inclusion can be seen in this image, represented by the reads between the first and middle exon (since the direction is left, as described within Table 1). These 14 reads are read-abundance-based, since they do not span the intron-exon junction. (B) depicts a closer view of the region shown in (A)—162905660-162905719. The dotted vertical black lines are centered upon the first base of the variant-containing exon. The thin lines in the middle panel that span the entire exon fragment are evidence of exon skipping. These 5 reads are split across the exon before and after the variant-containing exon, as seen in (A).

FIG. 5. Histogram of read-abundance-based intron inclusion with embedded Q-Q plots of the predicted leaky mutation (chr5:162905690G>T) within HMMR, as shown in FIG. 4. The arrowhead denotes the number of reads (14 in this case) in the variant-containing file, which is more than observed in the control samples (p=0.04).

FIG. 6. (A) depicts an inactivating mutation (chr10:89711873A>G) within the natural acceptor site of exon 6 (89711874-89712016) of PTEN. The dotted vertical black line denotes the location of the relevant splice site. The region displayed is 89711004-89712744 on chromosome 10. Many of the 32 exon skipping reads are evident, typified by the thin lines in the middle panel that span the entire exon. There is also a substantial amount of read-abundance-based intron inclusion, shown by the reads to the left of the dotted vertical line. Exon skipping was statistically significant (p<0.01), while read-abundance-based intron inclusion was not (p=0.53). Panels (B) and (C) depict an inactivating mutation (chr12:83359523G>A) within the natural donor site of exon 6 (83359338-83359523) of TMTC2. (B) depicts a closer view (83359501-83359544) of the region shown in (C) and only shows exon 6. Some of the 22 junction-spanning intron inclusion reads can be seen. In this case, all of these reads contain the mutation, shown by the adenine base in each read, between the two vertical dotted lines. (C) depicts a genomic region of chromosome 12: 83359221-83360885, TMTC2 exons 6-7. The variant occurs in the left exon. 65 read-abundance-based intron inclusion can be seen in this image, represented by the reads between the two exons. Panel (D) depicts a mutation (chr1:985377C>T) causing a cryptic donor to be activated within exon 27 (the second from left, 985282-985417) of AGRN. The region displayed is 984876-985876 on chromosome 1 (exons 26-29 are visible). Some of the 34 cryptic (junction-spanning) reads are portrayed. The dotted black vertical line denotes the cryptic splice site, at which cryptic reads end. The read-abundance-based intron inclusion, of which two reads are visible, was not statistically significant (p=0.68).

FIG. 7. IGV images and their corresponding histograms with embedded Q-Q plots depicting all six variant-containing files with a mutation (chr1:46726876G>T) which, in some cases, causes a cryptic donor to be activated within the intron between exons 7 and 8 of RAD54L. This results in the extension of the downstream natural donor (the 5′ end of exon 8). This gene has two transcript variants and the given exon numbers pertain to isoform a (reference sequence NM_003579). Only samples IV and V have statistically significant intron inclusion relative to controls. read-abundance-based intron inclusion can be seen in (A), between the two exons. The region displayed is on chromosome 1: 46726639-46726976. (B) depicts the corresponding histogram for the 15 read-abundance-based intron inclusion reads (p=0.05) that are present in sample IV. The intron-exon boundary on the right is the downstream natural donor. (C) typifies some of the 13 junction-spanning intron inclusion reads that are a direct result of the intronic cryptic site's activation. In these instances, reads extending past the intron-exon boundary are being spliced at the cryptic site, instead of the natural donor. In particular, samples IV and V both have a statistically significant numbers of such reads, 7 (p=0.01) and 5 (p=0.04), respectively. This is further typified by the corresponding histogram in (D). (C) focuses upon exon 8 from (A) and displays the genomic positions 46726908-46726957. Refer to the caption of FIG. 4 for IGV graphical element descriptions. In the histograms, arrowheads denote numbers of reads in the variant-containing files. The bottom of the plots provide p-values for each respective arrowhead. Statistically significant p-values and their corresponding arrowheads are denoted in grey shading.

FIGS. 8A, 8B, 8C, 8D and 8E are a list of splicing mutations in Cancer Genome Atlas samples that cause exon skipping due to creation or strengthening of exonic hnRNPA1 binding sites. For each panel, genes and corresponding mutations stipulating the first gene/mutation pair inclusive to the last gene/mutation pair are shown in panels (A) CDC14A, chromosome 1: 100964692C/T through MLXIP, chromosome 12:122622059A/G; (B) DDX55, chromosome 12:124102356C/T through CD97, chromosome 19:14517319C/G; (C) NOTCH3, chromosome 19:15303075A/G through FRG1, chromosome 4:190874240C/G; (D) RCHY1, chromosome 4:76416774A/C through OLFML2A, chromosome 9:127566488C/T; (E) SURF1, chromosome 9:136221752C/G through GOLM1, chromosome 9:88650378A/G. Each row indicates a different mutation, the gene which contains the mutation, the predicted change in R_{i,total} (total exon information content), the probability value calculated by Veridical for variant-induced exon skipping (to exclude the null hypothesis), the number of samples in which the mutation is present and those in which it is absent, the number reads which cause skipping of the exon containing the mutation in the samples in which it is present and absent, and whether or not the variant is a known single nucleotide polymorphism.

### DETAILED DESCRIPTION OF THE PREFERRED EMBODIMENTS

The Veridical method and software were developed to allow high-throughput validation of predicted splicing mutations using RNA sequencing data. Veridical requires at least three files to operate: a DNA variant file containing putative mRNA splicing mutations, a file listing of corresponding transcriptome (RNA-Seq) BAM files, and a file annotating exome structure. A separate file listing RNA-Seq BAM files for control samples (i.e. normal tissue) can also be provided. The capabilities of the method for mutations predicted in a set of breast tumours is demonstrated. Veridical compares RNA-Seq data from the same tumours with RNA-Seq data from control samples lacking the predicted mutation. However, in principle, potential splicing mutations for any disease state with available RNA-Seq data can be investigated. In each tumour, every variant is analyzed by checking the informative sequencing reads from the corresponding RNA-Seq experiment for non-constitutive splice isoforms, and comparing these results with the same type of data from all other tumour and normal samples that do not carry the variant in their exomes.

Veridical concomitantly evaluates control samples, providing for an unbiased assessment of splicing variants of potentially diverse phenotypic consequences. Note that control samples include all non-variant containing files (i.e. RNA-Seq files for those tumours without the variant of interest), as well any normal samples provided. Increasing the number of the set of control samples, while computationally more expensive, increases the statistical robustness of the results obtained.

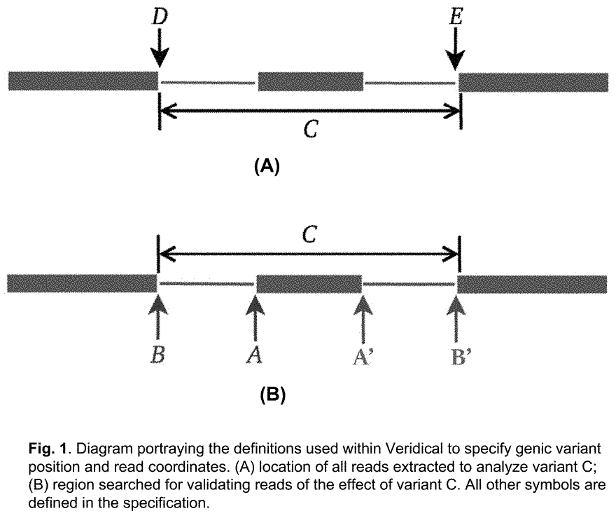

For each variant, Veridical directly analyzes sequence reads aligned to the exons and introns that are predicted to be affected by the genomic variant. We elected to avoid indirect measures of exon skipping, such as loss of heterozygosity in the transcript, because of the possibility of confusion with other molecular etiologies (i.e. deletion or gene conversion), unrelated to the splicing mutations. The nearest natural site is found using the exome annotation file provided, based upon the directionality of the variant, as defined within Table 1. The genomic coordinates of the neighboring exon boundaries are then found and the process iterates over all known transcript variants for the given gene. A diagram of this procedure is provided in FIG. 1. The variant location, C, is specifically referring to the variant itself. J_{c} refers to the variant-induced location of the predicted mRNA splice site, which is often proximate to, but distinct from the coordinate of the actual genomic mutation itself.

TABLE 1
Definitions used to determine the direction in which reads are checked.
A and B represent natural site positions, defined in FIG. 1(B).

Pertinent Splice Site

| A | B | Strand | Direction |
|---|---|---|---|
| Exonic | Donor^{α} | + | → |
| Exonic | Donor^{α} | − | ← |
| Intronic | Acceptor^{β} | + | ← |
| Intronic | Acceptor^{β} | − | → |

^{α}5′ splice site
^{β}3′ splice site

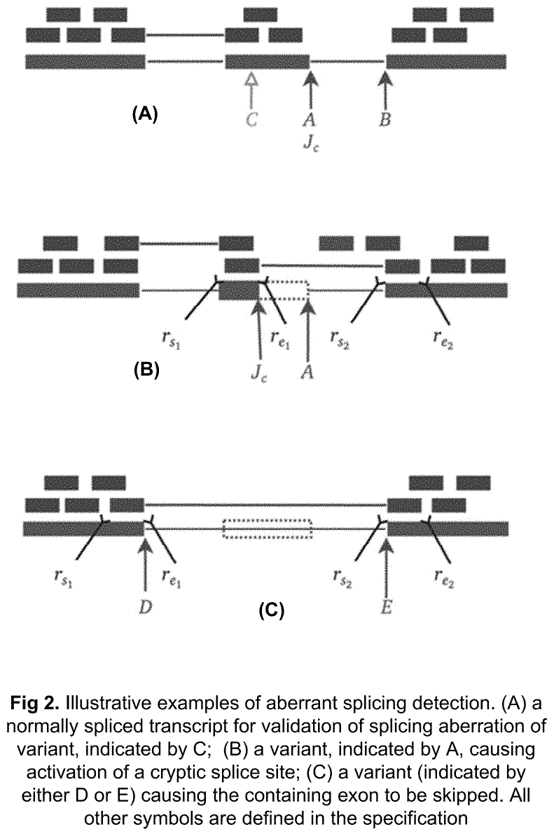

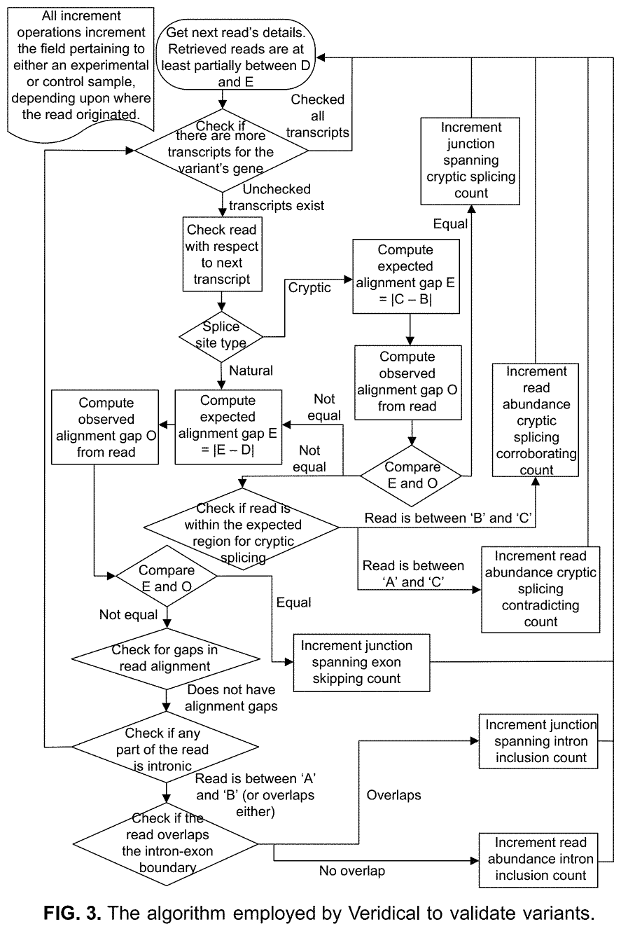

The program uses the BamTools API (Barnett et al. 2011) to iterate over all of the reads within a given genomic region across experimental and control samples. Individual reads are then assessed for their corroborating value towards the analysis of the variant being processed, as outlined in the flowchart in FIG. 3. Validating reads are based on whether they alter either the location of the splice junction (i.e. junction-spanning) or the abundance of the transcript, particularly in intronic regions (i.e. read-abundance). Junction-spanning reads contain DNA sequences from two adjacent exons or are reads that extend into the intron (Equation1(e)). These reads directly show whether the intronic sequence is removed or retained by the spliceosome, respectively. Read-abundance validated reads are based upon sequences predicted to be found in the mutated transcript in comparison with sequences that are expected to be excised from the mature transcript in the absence of a mutation (Equation 1(f)). Both types of reads can be used to validate cryptic splicing, exon skipping, or intron inclusion. A read is said to corroborate cryptic splicing if and only if the variant under consideration is expected to activate cryptic splicing. Junction-spanning, cryptic splicing reads are those in which a read is exactly split from the cryptic splice site to the adjacent exon junction (Equation1(a)). For read-abundance cryptic splicing, we define the concept of a read fraction, which is the ratio of the number of reads corroborating the cryptically spliced isoform and the number of reads that do not support the use of the cryptic splice site (i.e. non-cryptic corroborating) in the same genomic region of a sample. Cryptic corroborating reads are those which occur within the expected region where cryptic splicing occurs (i.e. spliced-in regions). This region is bounded by the variant splice site location and the adjacent (direction dependent) splice junction (Equation 1(a)). Non-cryptic corroborating reads, which we also term “anti-cryptic” reads, are those that do not lie within this region, but would still be retained within the portion that would be excised, had cryptic splicing occurred (Equation 1(b)). To identify instances of exon skipping, Veridical only employs junction-spanning reads. A read is considered to corroborate exon skipping if the connecting read segments are split such that it connects two exon boundaries, skipping an exon in between (Equation 1(c)). A read is considered to corroborate intron inclusion when the read is continuous and either overlaps with the intron-exon boundary (and is then said to be junction-spanning) or if the read is within an intron (and is then said to be based upon read-abundance). We only consider an intron inclusion read to be junction spanning if it spans the relevant splice junction, A. Equation 1(d) formalizes this concept. We occasionally use the term “total intron inclusion” to denote that any such count of intron inclusion reads includes both those containing and not containing the mutation itself. Graphical examples of some of these validation events, with a defined variant location, are provided in FIG. 2.

Formally, a given read is denoted by r, with start and end coordinates (rₛ, rₑ), if the read is continuous, or otherwise, with start and end coordinate pairs,

$$( {r}_{{s}_{1}} , {r}_{{e}_{1}} ) and ( {r}_{{s}_{2}} , {r}_{{e}_{2}} )$$

as shown in FIG. 2. Let £ be the length of the read. The set ζ denotes the totality of validating reads. The criterion for r∈ζ is detailed below. It is important to note that validating reads are necessary but not sufficient to validate a variant. Sufficiency is achieved only if the number of validating reads is statistically significant relative to those present in control samples. ζ itself is partitioned into three sets: ζ_{c}, ζₑ, and ζᵢ for evidence of cryptic splicing, exon skipping, and intron inclusion, respectively. We allow partitions to be empty. Let Jc denote the adjacent splice junction, and let B denote the downstream natural site, as defined by FIG. 2 and Table 1. Without loss of generality, we consider only the red (i.e. direction is right) set of labels within FIG. 1(B), as further typified by FIG. 2. Then the (splice consequence) partitions of ζ are given by:

r∈ζ_{c}⇔variant is cryptic ∧(rₛ₂−rₑ₁=B−J_{C}∨(rₛ>J_{C}∧rₑ<A)) (1a)

r∉ζ_{c}∧variant is cryptic ∧¬(rₛ₂−rₑ₁=B−J_{C})⇔r∈anti-cryptic (1b)

r∈ζₑ⇔(rₑ₁=D∧rₛ₂=E) (1c)

r∈ζᵢ⇔(A∈[rₛ,rₑ])∨((A∉[rₛ,rₑ])∧rₛ>A−l∧rₑ<B∧¬(A∈[rₛ,rₑ])) (1d)

We separately partition ζ by its evidence type, the set of junction-spanning reads, δ and read-abundance reads, α:

r∈δ⇔(A∈[rₛ,rₑ])∨(r∈ζ_{c}∧rₛ₂−rₑ₁=B−J_{C}) (1e)

r∈α⇔r∉δ (1f)

Once all validating reads are tallied for both the experimental and control samples, a p-value is computed. This is determined by computing a z-score upon Yeo-Johnson (YJ) transformed data. This transformation, shown in Equation 2, ensures that the data is sufficiently normally distributed to be amenable to parametric testing.

$$ψ ( x , λ ) = \{ \begin{matrix}\frac{{( x + 1 )}^{λ} - 1}{λ} & if x \geq 0 λ \neq 0 \\ log ( x + 1 ) & if x \geq 0 λ = 0 \\ - \frac{{( - x + 1 )}^{2 - λ} - 1}{2 - λ} & if x < 0 λ \neq 2 \\ - log ( - x + 1 ) & if x < 0 λ = 2\end{matrix} \qquad ( 2 )$$

The transform is similar to the Box-Cox power transformation, but obviates the requirement of inputting strictly positive values and has more desirable statistical properties. Furthermore, this transformation allowed us to avoid the use of non-parametric testing, which has its own pitfalls regarding assumptions of the underlying data distribution. We selected λ=½, because the untransformed output is skewed left, due to their being, in general, fewer validating reads in the control samples and the fact that there are, by design, vastly more control samples than experimental samples. We found that this value for λ generally made the distribution much more normal. Small departures from normality were not concerning, as a z-test with a large number of samples is robust to such deviations.

Thus, we can compute the p-value of the pairwise unions of the two sets of partitions of ζ, except the irrelevant ζₑ ∪α=0. We only provide p-values for these pairwise unions and do not attempt to provide p-values for the partitions for the different consequences of the mutations on splicing. Our previous work provides guidance on interpretation of splicing mutation outcomes (Rogan et al. 1998; Rogan et al. 2003; Mucaki et al. 2013; Shirley et al. 2013). Thus for ζₓ∈{ζ_{c}, ζₑ, ζᵢ}, let ϕ_{z}(z) represent the cumulative distribution function of the one-sided (right-tailed—i.e. P [X>x]) standard normal distribution. Let N represent the total number of samples and let V represent the set of all ζₓ validations, across all samples. Then:

$$μ = \frac{{\sum }_{j = 1}^{N} {V}_{j}}{N} σ = \sqrt{\frac{1}{N} {\sum }_{j = 1}^{N} {( {V}_{j} - \overline{V} )}^{2}}$$

$$z = \frac{\vert {Ϛ}_{i} \vert - μ}{σ} p = Φ ( ψ ( z , \frac{1}{2} ) )$$

with mean μ and standard deviation σ, z is the distance from μ for ζₓ reads, N is the total number of samples and V represents the set of all ζₓ validations, across all samples. The p-values given by Veridical are more robust when the program is provided with a large number of samples. The minimum sample size is dependent upon the desired power, a value, and the effect size (ES). The minimum samples size could be computed as follows: N=[σ² z²/ES²]. For α=0.05 and β=0.2 (for a power of 0.8), z=2.4865 for the one-tailed test. Then, N=[σ² 2.4865²/ES²]. Ideally, Veridical could be run with a trial number of samples. Then, one would compute effect sizes from Veridical's output. The standard deviation in the above formula could also be estimated from one's data, although it should be transformed using Yeo-Johnson before computing this estimation.

We elected to use RefSeq [19] genes for the exome an notation, as opposed to, the more permissive exome annotation sets, UCSC Known Genes [20] or Ensembl [21]. The large number of transcript variants within Ensembl, in particular, caused many spurious intron inclusion validation events. This occurred because reads were found to be intronic in many cases, when in actuality they were exonic with respect to the more common transcript variant. In addition, the inclusion of the large number of rare transcripts in Ensembl significantly increased program run-time and made validation events much more challenging to interpret unequivocally. The use of RefSeq, which is a conservative annotation of the human exome, resolves these issues. It is possible that some subset of unknown or Ensemble annotated intronic transcripts could be sufficiently prevalent to merit inclusion in our analysis. We perform the difficult task of deciding which of these transcripts would be worth using. Indeed, the task of confirming and annotating of such transcripts is already done by the more conservative annotation that we employ.

The method and software program outputs lists of all validated read counts across all categories for experimental samples and for the control samples. Probability values are shown in parentheses within the experimental table, which refer to the column-dependent (i.e. the read type is given in the column header) p-value for that read type with respect to that same read type in control samples. The program produces three files: a log file containing all details regarding validated variants, an output file with the programs progress reports and summaries, and a filtered validated variant file. The filtered file contains all validated variants of statistical significance (set as p<0.05, by default), defined as variants with one or more validating reads achieving statistical significance in a strongly corroborating read type. These categories are limited to all junction-spanning based splicing consequences and read-abundance total intron inclusion. For example, a cryptic variant for which p=0.04 in the junction-spanning cryptic column would meet this criterion, assuming the default significance threshold.

### EXAMPLES

The following examples are provided for purposes of illustration of embodiments of the present disclosure only and are not intended to be limiting. The reagents, chemicals, instruments and other materials are presented as exemplary components or reagents, and various modifications may be made in view of the foregoing discussion within the scope of this disclosure. Unless otherwise specified in this disclosure, components, reagents, protocol, and other methods used in the disclosure, as described in the Examples, are for the purpose of illustration only.

We demonstrate how Veridical is used to validate predicted splicing mutations in somatic breast cancer. Each example depicts a particular variant-induced splicing consequence, analyzed by Veridical, with its corresponding significance level. The relevant primary RNA-Seq data are displayed in IGV, along with histograms and Q-Q plots showing the read distributions for each example. The source data are obtained from breast carcinoma RNA and DNA sequences deposited in The Cancer Genome Atlas (TCGA; Koboldt et al. 2012). Tumour-normal matched DNA sequencing data from the TCGA consortium was used to predict a set of splicing mutations, and a subset of corresponding RNA sequencing data was analyzed to confirm these predictions with Veridical. Overall, 442 tumour samples and 106 normal samples were analyzed. Briefly, all variants used as examples came from running the matched TCGA exome files (to which the RNA-Seq data corresponds) through SomaticSniper (Larson et al. 2012) and Strelka (Saunders et al. 2012) to call somatic mutations, followed by the Shannon Human Splicing Pipeline (Shirley et al. 2013) to find splicing mutations, which served as the input to Veridical. Accordingly, the following examples demonstrate the utility of Veridical to identify potentially pathogenic mutations from a much larger subset of predicted variants.

### Example 1 Leaky Splicing Mutations

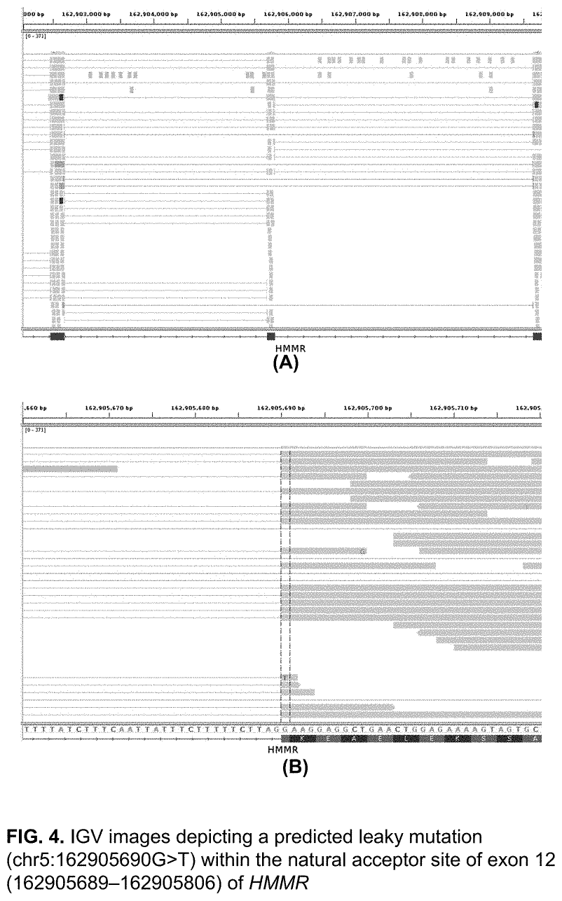

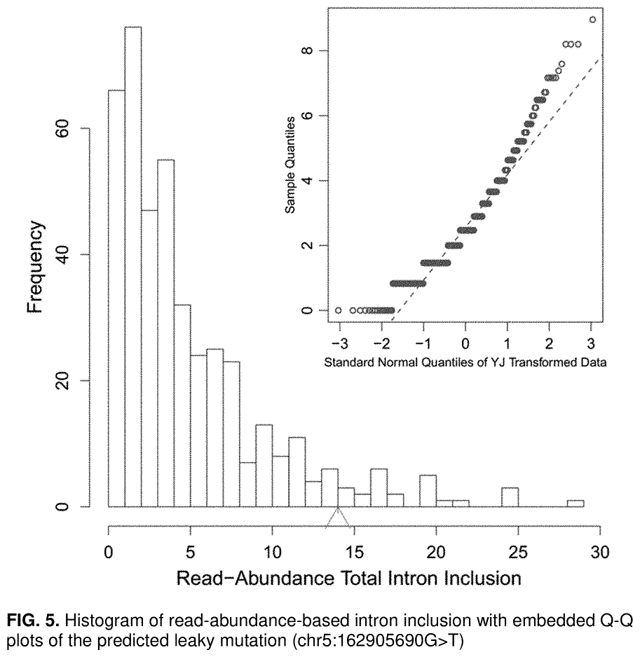

Mutations that reduce, but not abolish, the spliceosome's ability to recognize the intron/exon boundary are termed leaky³. This can lead to the mis-splicing (intron inclusion and/or exon skipping) of many but not all transcripts. An example, provided in FIG. 4, displays a predicted leaky mutation (chr5:162905690G>T) in the HMMR gene in which both junction-spanning exon skipping (p<0.01) and read-abundance-based intron inclusion (p=0.04) are observed. We predict this mutation to be leaky because its final Rᵢ exceeds 1.6 bits—the minimal individual information required to recognize a splice site and produce correctly spliced mRNA (Rogan et al. 2003). Indeed, the natural site, while weakened by 2.16 bits, remains strong—10.67 bits. This prediction is validated by the variant-containing sample's RNA-Seq data (FIG. 4), in which both exon skipping (5 reads) and intron inclusion (14 reads, 12 of which are shown, versus an average of 4.051 such reads per control sample) are observed, along with 70 reads portraying wild-type splicing. Only a single normally spliced read contains the G←T mutation. These results are consistent with an imbalance of expression of the two alleles, as expected for a leaky variant. FIG. 5 shows that for the distribution of read-abundance-based intron inclusion is marginally statistically significant (p=0.04).

### Example 2 Splice Site Inactivating Mutations

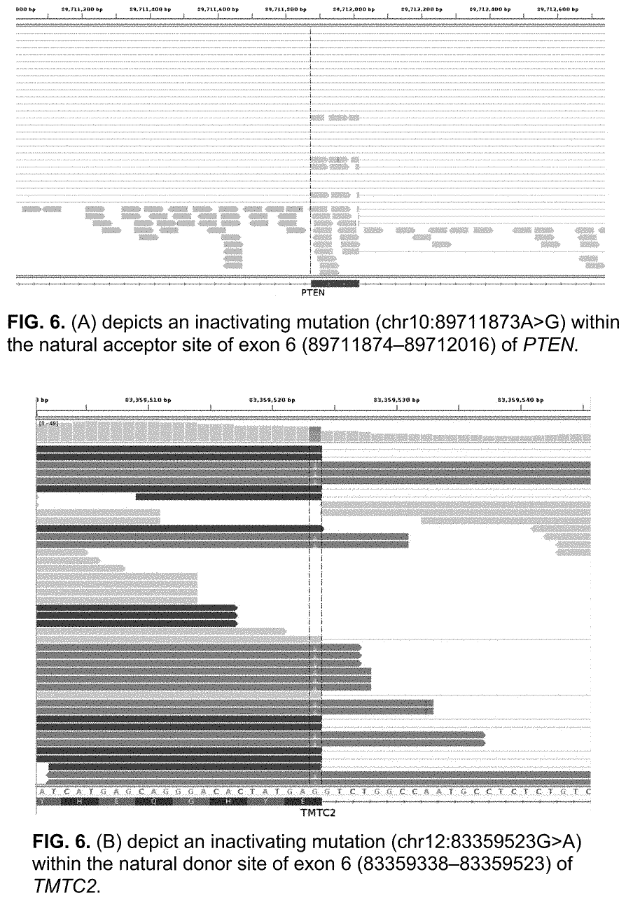

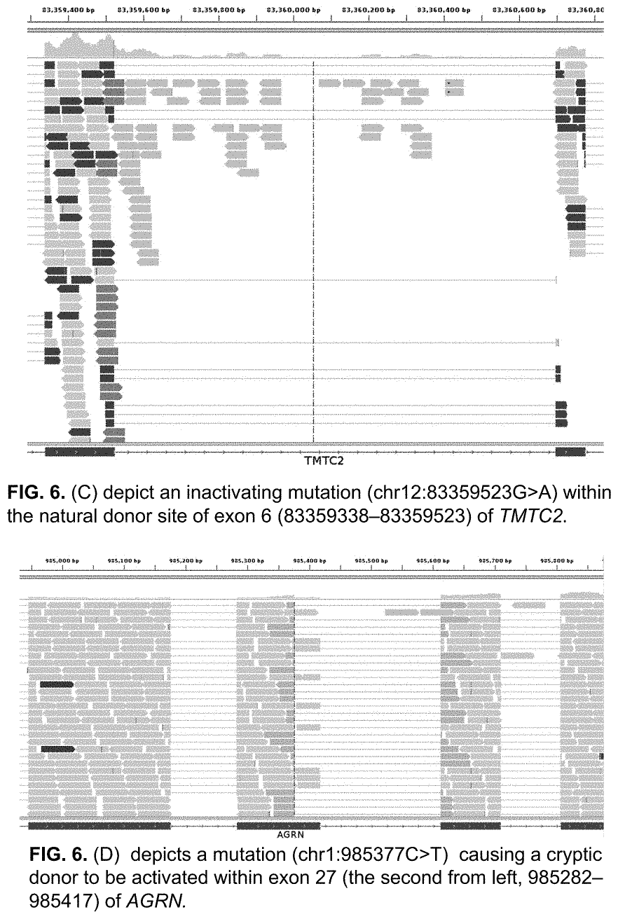

Variants that inactivate splice sites have negative final Rᵢ values (Rogan et al. 1998) with only rare exceptions (Rogan et al. 2003), indicating that splice site recognition is essentially abolished in these cases. We present the analysis of two inactivating mutations within the PTEN and TMTC2 genes from different tumour exomes, namely: chr10:89711873A>G and chr12:83359523G>A, respectively. The PTEN variant displays junction-spanning exon skipping events (p<0.01), while the TMTC2 gene portrays both junction-spanning and read-abundance-based intron inclusion (both splicing consequences with p<0.01). In addition, all intron inclusion reads in the experimental sample contain the mutation itself, while only one such read exists across all control samples analyzed (p<0.01). The PTEN variant contains numerous exon skipping reads (32 versus an average of 2.466 such reads per control sample). The TMTC2 variant contains many junction-spanning intron inclusion reads with the G←A mutation (all of its junction-spanning intron inclusion reads: 22 versus an average of 0.002 such reads per control sample). IGV screenshots for these variants are provided within FIG. 6. This figure also shows an example of junction-spanning cryptic splice site activated by the mutation (chr1:985377C>T) within the AGRN gene. The concordance between the splicing outcomes generated by these mutations and the Veridical results indicates that the proposed method detects both mutations that inactivate splice sites and cryptic splice site activation.

### Example 3 Cryptic Splicing Mutations

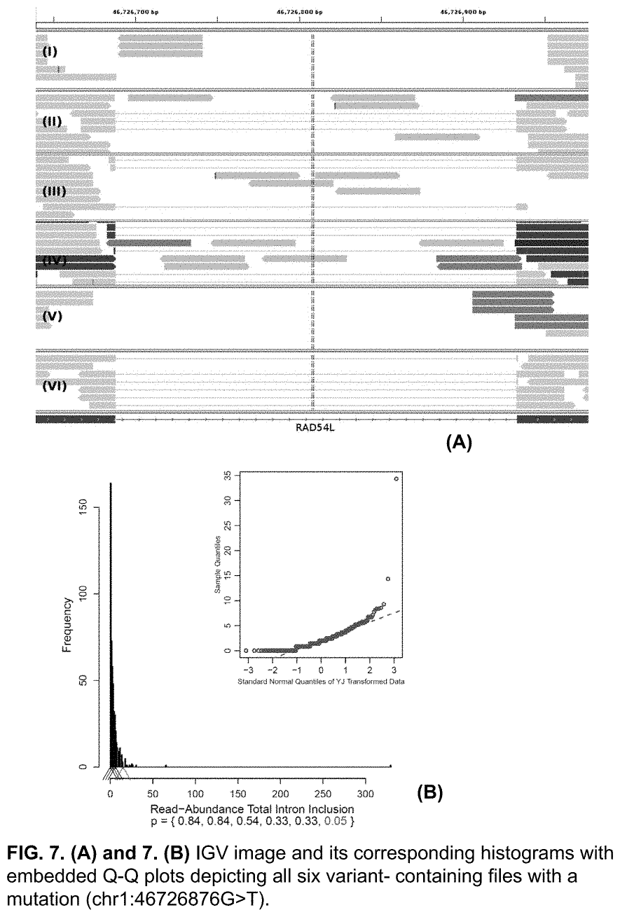

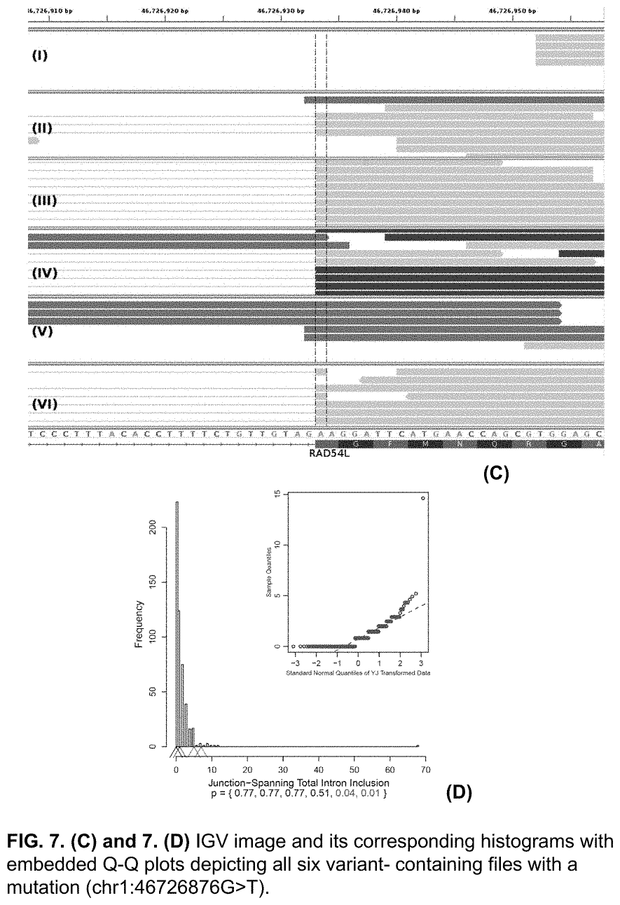

Recurrent genetic mutations in some oncogenes have been reported among tumours within the same, or different, tissues of origin. Common recurrent mutations present in multiple abnormal samples are recognized by Veridical. This avoids including a variant-containing sample among the control group, and outputs the results of all of the variant-containing samples. A relevant example is shown in FIG. 7. The mutation (chr1:46726876G>T) causes activation of a cryptic splice site within RAD54L in multiple tumours. Upon computation of the p-values for each of the variant-containing tumours, relative to all non-variant containing tumours and normal controls, not all variant-containing tumours displayed splicing abnormalities at statistically significant levels. Of the six variant-containing tumours, two had significant levels of junction-spanning intron inclusion, and one showed statistically significant read-abundance-based intron inclusion.

### Example 4 Generation of Information Theory-Based Models of mRNA Splicing Regulatory Proteins

Successful implementation of the information theory-based exon definition model is dependent on the quality of the data used to create the information weight matrices that locate and define the strengths of binding sites. Splice junctions are precisely defined and experimentally validated.

CLIP-seq libraries for hnRNP A1 (Huelga et al., 2012), and other splicing regulatory binding sites were used to derived information-theory based position weight matrices (PWM). PoWeMaGen software, which uses Bipad (Bi and Rogan, 2004) to generate a minimum entropy alignments, generates a series of potential binding site models over a range of input parameters. To mitigate against phasing the alignment on natural splice sites instead of adjacent hnRNP A1 binding sites, models were built from shorter sequences, ranging in lengths from 18-25nt. The optimal model was determined by maximizing incremental information by varying binding site length (6-10nt), number of Monte Carlo cycles (250-5000), and allowing either zero or only one site per sequence (OOPS). The model with the highest average information used a maximum fragment length of 18nt, 1000 Monte Carlo cycles, OOPS, and a single block binding site length of 6nt.

CLIP-seq data were used to compute PWMs for the following RNA binding proteins that participate in the mRNA splicing reaction and/or in exon definition:

### TIA1

### Ri(b,l) Length of PWM—12nt

Monte Carlo cycles—1000
ZOOPS (Zero Or One site Per Sequence)—On

### Source:

Wang Z, Kayikci M, Briese M, Zarnack K, Luscombe N M, Rot G, Zupan B, Curk T, Ule J. iCLIP predicts the dual splicing effects of TIA-RNA interactions. PLoS Biol. 2010 Oct. 26; 8(10):e1 000530

### PTB

### Ribl Length—6nt, 10nt

Monte Carlo cycles—250, 1000

### ZOOPS—On, On

### Source:

Xue Y, Ouyang K, Huang J, Zhou Y, Ouyang H, Li H, Wang G, Wu Q, Wei C, Bi Y, Jiang L, Cai Z, Sun H, Zhang K, Zhang Y, Chen J, Fu X D. Direct conversion of fibroblasts to neurons by reprogramming PTB-regulated microRNA circuits. Cell. 2013 Jan. 17; 152(1-2):82-96.

### HuR

### Ribl Length—7nt

Monte Carlo cycles—250
ZOOPS—Off (ON ribl is also available, but is very similar)

### Source:

Kishore S, Jaskiewicz L, Burger L, Hausser J, Khorshid M, Zavolan M.
A quantitative analysis of CLIP methods for identifying binding sites of RNA-binding proteins. Nat Methods. 2011 May 15; 8(7):559-64.

Each model or PWM was validated with a set of independently published binding sites and if available, mutations in those binding sites. As an example, validation of hnRNP A1 binding sites and mutations are presented, however the same approach was used for the other PWMs. A coding sequence mutation in the ETFDH gene c.158A>G creates a 5.9 bit hnRNP A1 site and increases exon skipping (Olsen et al., Hum. Mutation, 2013). BRCA2 mutation c.8165C>G similarly increases skipping and is predicted to create a 6.2 bit site (Liede et al., 2002). In contrast, the variant c.1161A>G in ACADM decreases exon skipping of exon 11 by reducing the strength of an hnRNP A1 site (6.1 to 1.4 bits). The model also predicted the existence of two strong hnRNP A1 binding site in a region of ATM shown to bind to the splicing regulator (Pastor and Pagani, 2011).

The effects of mutations at hnRNP A1 sites on exon definition were determined from the total information content (R_{i,total}), by incorporating changes in the strengths of these sites, corrected for the gap surprisal, which represents the distance between the hnRNP A1 site and the natural splice site. Gap surprisal values were determined by scanning the genome for hnRNP A1 sites with the PWM, and then determining the frequency of each interval length between known natural sites and the nearest hnRNP A1 site, separately for exons and introns. Differences between the natural and mutated exon R_{i,total} values correspond to changes in the abundance of the respective isoforms, and can predict exon skipping. The calculation is carried out by the Automated Splice Site and Exon Definition Analysis Server (ASSEDA; Mucaki et al. 2013). Exon definition analysis in ASSEDA was validated for a set of mutations that affect hnRNP A1 binding site strength. BRCA2 variant c.8165C>G decreases the R_{i,total} from 13.5 to 3.2 bits and results in exon skipping. ACADM variant c.1161A>G, which reduces exon skipping, increases the R_{i,total} from 18.5 to 20.1 bits.

Table 2 summarizes the validation results for models derived CLIP Seq data by evaluating published, peer reviewed binding sites in individual genes.

TABLE 2
Summary of validation results

| RNA | Binding |
|---|---|
| binding | sites |
| protein | Validated |
| 9G8 | 1 of 4 |
| TIA1 | 7 of 7 |
| PTB | 4 of 4 |
| HuR | 6 of 6 |
| hnRNPA1 | 3 of 3 |
| hnRNPC | 3 of 4* |
| hnRNP | 0 of 1 |
| A2/B1 | |
| hnRNP F | 1 of 2 |
| hnRNP U | 1 of 1 |

Valation of the model is measured by the success rate of binding site models to predict published binding sites in the sequence interval described in the literature publication (successfully detected sites vs total number of binding sites tested). The exact location for the binding site was not always known from the publication, and in those cases, we sought to detect the strongest sites with the highest Rᵢ values within that region, as described below. The results of optimal model construction include sequences logos and Rᵢ(b,l) matrices, and links to the papers reporting the binding sites, among others.

Based on these validation results, the PTB and hnRNP A1 models have been qualified for mutation analysis. The information contents generated from these PWMs are completely concordant with the published results for all known binding sites, and their motifs (as depicted by the corresponding sequence logos) have a distinct, complex pattern.

The TIA1, HuR and hnRNP C model validation was also quite succesful, but these PWMs consist of low complexity, T-rich motifs (based on DNA sequence, in RNA, which the protein binds to, these are Uridine) that have lower specificity than the PTB and hnRNP A1 binding sites. For TIA1 and HuR, this pyrmidine-rich region is where binding is expected. There have been concerns that these models will positively identify a binding site in nearly any poly-T rich region. As an example, one can refer to the HuR model, in which almost all information is derived from poly-T.

Summary of data on RNA binding protein motifs that are involved in mRNA splicing obtained by entropy minimization of Clip-Seq data is provided in the following text.

### TIA1/TIAL1

TIA-1 promotes U1 snRNP binding to the 5′ splice site of intron 6 of FAS. Exonic TIA-1 binding to Uridine-rich sequences mediate repression by PTB at the acceptor (3′) site, promoting exon skipping (Izquierdo et al., Molecular Cell, 19: 475-484, 2005). This model does correctly recognize exon 3′ terminus at position 573, 3.2 bit site at 576, 4.9 bit site at 596, and a 3-4 bit cluster from 600-602.

The RNA-binding protein TIA-1 preferentially enhances the use of 5′ splice sites linked to IAS1 (for example, the alternative K-SAM exon in FGFR2 gene)—which are then activated by overexpression of TIA1. See Del Gatto-Konczak et al. Mol Cell Biol. 2000; 20(17):6287-99.

Approximately 20 nucleotides beyond the end of the K-SAM exon, information analysis predicts large cluster of strong binding sites (chromosome 10:123278160-123278310), associated with a long polyT/poly A track. This result is consistent with the well described property of TIA-1 binding to polyAU-rich domains of RNA.

| Chr. Coord. | Ri value |
|---|---|
| 123278167 | 5.669410 |
| 123278168 | 10.217979 |
| 123278169 | 2.813830 |
| 123278170 | 5.144820 |
| 123278171 | 4.534150 |
| 123278172 | 8.654270 |
| 123278173 | 1.410610 |
| 123278177 | 4.872140 |
| 123278178 | 1.938000 |
| 123278179 | 5.716410 |

In the SMN2 gene, exon 7 inclusion is regulated by TIA-1 interacting with the U1 SNRNP. See N. Singh and R. Singh, Alternative splicing in spinal muscular atrophy underscores the role of an intron definition model, RNA Biol. 2011 July-August; 8(4): 600-606. There are two validated TIA-1 sites within the interval (chr5: 69, 372,420-69,372,490).

| Chr. Coord. | Ri value |
|---|---|
| 69372436 | 6.438010 |
| 69372437 | 1.917100 |
| 69372438 | 3.805560 |
| 69372439 | 4.751070 |
| 69372441 | 2.209620 |
| 69372456 | 2.445030 |
| 69372463 | 3.158220 |
| 69372466 | 2.991800 |
| 69372469 | 1.997720 |
| 69372472 | 4.344520 |
| 69372473 | 3.055380 |
| 69372474 | 4.637970 |
| 69372475 | 9.499431 |
| 69372477 | 2.657180 |
| 69372480 | 1.036970 |
| 69372482 | 6.704550 |
| 69372483 | 1.218490 |
| 69372490 | 2.263090 |

In all 3 instances of valid binding sites in SMN2, a site was found (bolded). The sites exceed 5 bits. Interestingly, the 9.5 bit site is in a region, where a binding site is expected based on experimental data, but has not been localized (described as “ELEMENT 2” in the publication).

In summary, the TIA-1 model detected strong sites, but weak false positives were also present, as a result of the promiscuity of A/T rich regions being flagged. In order to eliminate false positive binding sites, the TIA1 model is preferably used in combination with a second motif for a distinct RNA binding protein, which is known to interacts with, for example, PTB. The combined motif could be computed as a R_{i,total} value, based on the strengths of each sites, and the gap surprisal distribution which relates both sites.

Although it is quite accurate, the hnRNP C model confirmed 3 of 4 published binding sites all from papers that demonstrated binding within a 20-70 nt long region, none of which described the precise location of the binding sites. The one that failed was the only one that involved a mutation which supposedly abolished an hnRNP C site, which was not detected with either of the hnRNP C models developed.

Models for both hnRNP F and hnRNP U result in high bit values for natural splice sites (both donors and acceptors). The ‘CAG’ pattern in the sequence logo is quite obvious. The possibility cannot be eliminated that the entropy minimization is biasing toward more conserved natural sites, which “contaminate” these sequences due to their proximity to the hnRNP sites. Furthermore, hnRNP F binding sites are known to have a GGG motif, which is absent from any model built from the hnRNP F data.

Hu proteins inhibit splicing by binding to intronic recognition sequences adjacent to exon 23a of NF1 (HuB, HuC, and HuD) and adjacent TIA1 sites promote recognition of the donor splice site by U1 SNRNP. See Zhu, et al. Mol Cell Biol. 2008 February; 28(4): 1240-1251. Within chr17:29,579,900-29,580,100, TIA-1 sites are present at:

| Chr. Coord. | Ri value (bits) |
|---|---|
| 29580015 | 3.791960 |
| 29580029 | 7.952610 |

A series of Hu protein binding sites has been predicted at a weak donor site in the PLOD2 gene (chromosome 3:145,795,600-145,795,750). See Yeowell, Heather N, Walker, Linda C, Mauger, David M, Seth, Puneet, Garcia-Blanco, Mariano A. TIA

Nuclear Proteins Regulate the Alternate Splicing of Lysyl Hydroxylase 2, Journal of Investigative Dermatology (2009) 129,1402-1411.

| Chr. Coord. | Ri value (in bits) |
|---|---|
| 145795604 | 6.539410 |
| 145795605 | 2.437480 |
| 145795607 | 5.573260 |
| 145795609 | 4.282010 |
| 145795610 | 3.696390 |
| 145795611 | 6.333310 |
| 145795612 | 0.722530 |
| 145795613 | 8.514270 |
| 145795614 | 6.387630 |
| 145795615 | 6.179630 |
| 145795616 | 7.204071 |
| 145795617 | 8.928380 |
| 145795618 | 0.453510 |
| 145795619 | 7.776460 |
| 145795620 | 4.122941 |
| 145795621 | 4.207820 |
| 145795622 | 9.756490 |
| 145795624 | 5.764780 |
| 145795625 | 3.915710 |
| 145795626 | 6.074350 |
| 145795627 | 0.233480 |
| 145795628 | 6.985560 |
| 145795629 | 2.751471 |
| 145795630 | 7.838311 |
| 145795631 | 8.452850 |
| 145795632 | 10.973180 |
| 145795633 | 7.993841 |
| 145795634 | 6.453230 |
| 145795635 | 7.710070 |
| 145795636 | 1.090840 |
| 145795638 | 3.965630 |
| 145795640 | 9.942340 |
| 145795641 | 8.432720 |
| 145795642 | 4.729580 |
| 145795643 | 2.373280 |
| 145795644 | 3.849880 |
| 145795645 | 5.682571 |

PTB.

Two different models were computed for PTB, which differ only by the length of the binding sites. The 6SB model is preferred based on published studies on PTB. However the 6SB model may truncate the site, which is one of the reasons why the 10SB model was also derived.

As described previously by Izquierdo et al. (2005), PTB represses inclusion of the exon 6 in FAS, which was described for TIA1 (although the PTB site is in exon 6). The interval containing the PTB binding sites span the interval chromosome 10:90,770,450-90,770,649. With the 6SB model, several potential binding sites were detected in this interval (the strongest sites are bolded).

| Chr. Coord. | Ri value (bits) |
|---|---|
| 90770505 | 1.103880 |
| 90770512 | 3.856850 |
| 90770517 | 1.824200 |
| 90770535 | 4.674070 |
| 90770543 | 4.955421 |
| 90770556 | 3.293820 |
| 90770564 | 3.055950 |
| 90770578 | 0.367950 |
| 90770582 | 3.384770 |
| 90770589 | 1.924930 |

The two strongest predicted binding sites contain the “URE6 element” described in the publication, and contain PTB “consensus” sequence, UCUU. Using the 10SB model, the corresponding sites are 2.94 and 1.13 bits, respectively, with the 3.3 bit site at 90770556 strengthening it from 3.3 to 4.5 bits.

PTB binding to the CHRNA gene has also been reported in the region, chromosome 2: 175622750-17562290 (Rahman M A, Masuda A, Ohe K, Ito M, Hutchinson D O, Mayeda A, Engel A G, Ohno K. HnRNP L and hnRNP LL antagonistically modulate PTB-mediated splicing suppression of CHRNA1 pre-mRNA. Sci Rep. 2013 Oct. 14; 3:2931.). The 7.3 bit site at position 175622764 is described in the publication (Bian Y, Masuda A, Matsuura T, Ito M, Okushin K, Engel A G, Ohno K. Tannic acid facilitates expression of the polypyrimidine tract binding protein and alleviates deleterious inclusion of CHRNA1 exon P3A due to an hnRNP H-disrupting mutation in congenital myasthenic syndrome. Hum Mol Genet. 2009 Apr. 1; 18(7):1229-37). However, the present disclosure provides a 5.8 bit site close to the branch point.

PTB also binds to both ends of exon 9 of the gene, CAPZB. Downstream of the exon near position 19669210, there is a 3.7 bit site situated between two ACUAA elements (with the 1 Ont long ribl, 2.2 bits with the 6SB model), which are recognized by the RNA binding protein, Quaken. No other predicted sites exist in this region. Upstream of the exon around position 19669400, the published study is less precise about the location of the PTB site. The model of the instant disclosure predicted several potential sites in this region, including a 6.7 bit site ˜40nt downstream of the exon and a 4.4 bit site ˜10nt downstream.

HuR/ELAVL1

HuR (or ELAVL1) regulates inclusion of an exon in the FAS gene, though there is evidence to suggest it is interacting with URE6. HuR is predicted to bind at several locations across exon 6 and upstream in intron 5 (Izquierdo J M. Hu antigen R (HuR) functions as an alternative pre-mRNA splicing regulator of Fas apoptosis-promoting receptor on exon definition. J Biol Chem.2008 Jul. 4; 283(27)19077-84). The region upstream of the exon (chr10:90,770,450-90,770,649) has a cluster of strong HuR binding sites:

| Chr. Coord | Ri value (in bits) |
|---|---|
| 90770471 | 6.351841 |
| 90770472 | 8.330290 |
| 90770475 | 7.383730 |
| 90770477 | 5.040200 |

Within the exon, there is only a single cluster of strong binding sites, which coincides with the location of the URE6 element, as indicated in the article:

| Chr. Coord | Ri value (in bits) |
|---|---|
| 90770535 | 3.071350 |
| 90770538 | 4.882600 |
| 90770541 | 4.882600 |
| 90770542 | 2.393560 |
| 90770543 | 9.590730 |

HuR exhibits documented binding to the ATM gene. However, binding did not impact the mRNA splicing profile of this gene. There are 9 consecutive thymine residues, which results in a set of strong binding sites, corresponding to the interval described in the paper (˜80 nucleotides in length).

| Chr. Coord | Ri value (in bits) |
|---|---|
| 108141430 | 3.633660 |
| 108141431 | 7.772871 |
| 108141432 | 12.418920 |
| 108141433 | 12.418920 |
| 108141434 | 12.418920 |
| 108141435 | 2.882740 |

In Hu et al. Mol Cell Biol. 2008 February; 28(4): 1240-1251 (cited previously for TIA-1), the authors indicate that multiple Hu proteins bind to exon 23a of NF1. Our HuR model predicts a number candidate binding sites in this region.

| Chr. Coord. | Ri (in bits) |
|---|---|
| 29579831 | 2.263210 |
| 29579832 | 4.191080 |
| 29579833 | 3.633660 |
| 29579834 | 7.772871 |
| 29579835 | 2.882740 |
| 29579836 | 0.863631 |
| 29579837 | 7.102510 |

In the publication, the TIA1 site is described as adjacent to a Hu binding site downstream of the exon. 9.3 and 5.5 bit HuR binding sites were found (at pos. 29580034-35) immediately upstream and one 7.0 bit HuR site at pos. 29580047 downstream of the TIA1 site.

hnRNP A1

The following study shows that hnRNAP A1 regulates splicing of the ATM gene (Pastor T, Pagani F. Interaction of hnRNPA1/A2 and DAZAP1 with an Alu-derived intronic splicing enhancer regulates ATM aberrant splicing. PLoS One. 2011; 6(8):e23349) and binds within a 35 nucleotide interval circumscribing position 108141450.

| Chr. Coord | Ri value (in bits) |
|---|---|
| 108141439 | 5.652870 |
| 108141457 | 1.664050 |
| 108141469 | 4.653870 |

A sequence variant creates an hnRNP A1 site within ETFDH (also HNRNP A2/B1 and H). See Olsen R K, Brener S, Sabaratnam R, Doktor T K, Andersen H S, Bruun G H, Gahrn B, Stenbroen V, Olpin S E, Dobbie A, Gregersen N, Andresen B S. The ETFDH c.158A>G Variation Disrupts the Balanced Interplay of ESE- and ESS-Binding Proteins thereby Causing Missplicing and Multiple Acyl-CoA Dehydrogenation Deficiency. Hum Mutat. 2013 Oct. 7. doi: 10.1002/humu.22455.

This exonic variant at 159601742 was analyzed by information analysis to assess the predicted change in hnRNP A1 site strength. This exon itself is non-constitutive, and it is predicted that this variant increases the hnRNP A1 splicing suppressor strength, thereby increasing exon skipping (hnRNP A1 site at pos. 159601740, with R_{i,initiai}=−11.16->R_{i,final}=5.94 bits).

In addition, a weak hnRNP H binding site is created (0.62 bits at pos.15961742), and another pre-existing site is strengthened (3.79->4.03 bits at pos. 15960173). An preexisting 6.9 bit site 17nt downstream of the 4.0 bit site was also observed.

Analysis of this mutation with the hnRNP A2/B1 exon silencer model below did not detect any overlapping or novel binding sites.

hnRNP A2B1

A different variant in another gene was found to alter strengths in splicing regulatory sequences, bound by SFSR1 and hnRNP A1, in an alternative exon of the ACADM gene (Bruun G H, Doktor T K, Andresen B S. A synonymous polymorphic variation in ACADM exon 11 affects splicing efficiency and may affect fatty acid oxidation. Mol. Genet Metab. 2013 September-October; 110(1-2):122-8). c.1161A>G improves exon 11 inclusion in ACADM. The A form has been experimentally shown to increase hnRNP A1 binding, whereas the G allele binds SFSR1 (SF2/ASF) with higher affinity. Our predictions follow the experimental results precisely (hnRNP A1 at coordinate 76227021 is reduced in strength 6.12->1.37 bits, and SFSR1 (SF2/ASF) is increased −3.08->2.77 bits.

### Example 5 Application of R_{i,total} to Splicing Regulation—Experimental Validation of BRCA1 and BRCA2 Gene Mutations Predicted by Exon Definition Analysis

Numerous unclassified variants (UVs) have been identified in splicing regions of disease-associated genes and their characterization as pathogenic mutations or benign polymorphisms is crucial for the understanding of their role in disease development. The number of these alterations has increased considerably as a consequence of next generation sequencing analyses and confounds distinction of disease variants.

The aim of the present study was to assess the splice isoforms predicted by ASSEDA, through qPCR-based analyses. Where mRNA was available, we compared cryptic isoforms computed by exon definition analysis and their predicted abundance to results from semi quantitative RT-PCR and quantitative RT-PCR studies. Twenty-four UVs in BRCA genes were previously characterized by conventional end-point Reverse Transcriptase-PCR (RT-PCR) [1]. Nineteen splicing mutations and 5 non-spliceogenic base changes were observed. All variants were re-evaluated using ASSEDA (Mucaki et al. 2013). The value of the Window Range (i.e., the region before and after the base where the mutation takes place and where the information content of sites is calculated) was set to 450 nt.

The qPCR assays were performed using the KAPA SYBR FAST Universal qPCR kit (KAPA BIOSYSTEMS) and examined on an Eco Real-Time PCR System (Illumina). The level of expression of each isoform was measured relative to the level of expression of the same isoform in a reference sample. In addition, the level of expression of each isoform considered in the assay was normalized to the expression of CCDC137, as a reference gene. For each assay, uniform length amplicons were generated from reverse transcripts using isoform-specific splice junction primers. For the BRCA1 c. 4987-1G>A the normal transcript, the Δexon17 isoform and the transcript derived from the partial retention of intron 16 (187 bp at the 3′-end) were analyzed. For the BRCA1 c.5278-2delA the normal transcript, the Δexon21 isoform and the transcripts derived from the partial skipping of exon 21 (8 bp at the 5′-end) and the partial retention of intron 20 (51 bp at the 3′-end) were verified. In both analyses, a fragment spanning BRCA1 exon 8-9 junction was generated to serve as an internal reference.

ASSEDA detected all splicing mutations (n=19) and 9 of 11 cryptic isoforms observed in UV carriers (Table 1). Non-spliceogenic variants (n=5) did not exhibit significant changes in exon information. Cryptic isoforms of lower abundance not seen in previous analyses were also predicted (between 0 and 4 transcripts per mutation). Verification of these predictions by qPCR is currently ongoing. At present, the BRCA1 c. 4987-1G>A and c.5278-2delA mutations were analyzed. The full-length and the Δexon17 isoforms for the BRCA1 c. 4987-1G>A mutation and the full-length, the Δexon21 and the Δexon21q isoforms for the 5278-2delA were confirmed. However, additional low abundance isoforms predicted by ASSEDA were not observed in qPCR experiments, as expected.

Based on these results, it is concluded that information theory-based exon definition comprehensively detects the experimentally-verified repertoire of mutant isoforms by end point RT-PCR in carriers of the investigated UVs. Preliminary results show that qPCR analyses can determine which of the many potential intronic cryptic splice sites that are predicted by ASSEDA are potentially relevant and which ones can be dismissed as being irrelevant to pathogenicity.

The loss of exon identity due to the combined activation of binding sites associated with silencing of exon recognition and loss of binding sites recognized by exon enhancers has been shown. See Sterne-Weiler T, Howard J, Mort M, Cooper D N, Sanford J R, Loss of exon identity is a common mechanism of human inherited disease. Genome Res. 2011 October; 21(10):1563-71. However, although Sterne-Weiler et al. implicated specific hexamer sequences as contributing to exon skipping, and the splicing factors PTB and SRp20 in regulation of exon skipping, the context of these sequences with respect to their distance to the adjacent constitutive splice sites was not addressed or considered.

U.S. Pat. No. 8,361,979 B2 describes a method for inducing exon skipping by targeting oligonucleotide sequences to Serine-Arginine rich proteins that promote exon inclusion. However, the method of the '979 patent does not recognize the role that hnRNP A1 plays in proofreading of exon boundaries, nor does it consider that the proximity between this splicing regulatory sequence and the adjacent constitutive splice site is important for exon definition (i.e. Targeting neighboring and distant binding sites is likely to have different effects), and does not transform that distance into units of bits, i.e. Gap surprisal, so as to compute R_{i,total}, the method described in the instant invention for predicting exons that are recognized and processed in unspliced heteronuclear RNAs.

### Example 6 Exon Definition Analysis Reveals a Previously Unrecognized, but Common Mechanism of Exon Skipping Based on hnRNP A1 Cryptic Site Generation

Recursive stop-gain mutation c.5791C>T (rs144567652) in FANCM abolishes exon definition, inducing exon skipping and is a risk factor for familial breast cancer. The c.5791C>T mutation originates a stop codon at residue 1931 generating the loss of 118 amino-acids from the FANCM C-terminus that destroys the functional domain that mediates the interaction with FAAP24 (Ciccia et al. 2007) and DNA translocation (Rosado et al. 2009). However, functional analyses in lymphoblastoid cell lines obtained from two mutation carriers resulted a very low level of the mutated mRNA, suggesting that the c.5791C>T has a loss of function effect. This result was unexpected because this mutation occurs in the penultimate exon of the gene, where nonsense mediated decay, the predominant cellular mechanism of mRNA surveillance of premature stop codons, is not expected to cause significant mRNA degradation due to its close proximity to the 3′ untranslated region of the mRNA (Shoemaker E and Green R, Nature Struct. & Mol. Biol. 19: 594-601, 2012).

Information theory-based mutation analysis was used to assess the impact of the variant on splicing regulatory binding sites that regulate definition of the exon. The mutation is predicted to create an overlapping 4.6 bit hnRNP A1 binding site (c.5790_5795; Mucaki et al. 2013), which completely suppresses normal exon recognition (R_{i,total}: 3.4 (C) −>−2.6 (U) bits, inactivating exon recognition and results in complete exon skipping. The novel hnRNP A1 binding site sequence is frequently present in sites crosslinked to hnRNP A1 protein (Huelga et al. 2012). The frequencies of the normal and mutated FANCM hnRNPA1 sites from the sequences that were used to build the model for the present disclosure shows 140431 binding sites total in the model. The wild type site (CCGAAU) was not present, which is consistent with its negative Ri value. However, the mutant site CUGAAU was present 716 times in set of binding sites crosslinked to the protein. These are experimental data from crosslinking experiments using an antibody against hnRNP A1 to pull down these sequences. The reason why exon skipping occurs is related to one of the key functions of hnRNP A1. HnRNP A1 proofreads U2AF binding at the 3′ splice site. It also directly interacts with the 5′ splice site. See N. R. Zearfoss, E S. Johnson and S P. Ryder, hnRNP A1 and secondary structure coordinate alternative splicing of Mag, RNA (2013) 19: 948-957. For this protein binding site (Tavenez et al. 2012), exonic hnRNP A1 sites distant from known splice sites are very rare in the transcriptome (FIG. 2, which is consistent with abrogration of exon definition and exon skipping (Olsen et al. 2013). Skipping of exon 22 prematurely terminates translation after incorporating 11 frameshifted residues from exon 23, and the loss of 143 amino-acids from the FANCM C-terminus (p.Glyl906Alafs11*). This recursive property which introduces a premature stop codon further upstream of p.R1931X ensures that the mutant FANCM is incapable of complexing with FAAP24 or binding DNA.

The opal codon in FANCM contained the core sequence of the novel hnRNP A1 site (positions 1-3 of FIG. 13) in FANCM and the amber codon also contains conserved nucleotides in this binding site (positions 0-2 of FIG. 13). It appears that creation at hnRNP A1 coincident stop codons is a general mechanism to ensure exon skipping at these sites. Because the Ri(b,l) weight matrix that other CGA>TGA (Arg>Ter) mutations would be expected to activate hnRNP A1 sites, the National Center for Biotechnology Information's ClinVar database was searched with search term: (“stop gain”[Molecular consequence]) and all of the Arg>Ter mutations were analyzed with the instant invention. Arg>Ter is a very common stop-gain mutation in this database, which consists of published mutations as well as those contributed by clinical molecular diagnostic laboratories. More than 80% of the mutations analyzed create an hnRNP A1 site exceeding 3.5 bits in strength (in some cases, creating 2 sites). If the site is more than 40 nucleotides distant from the adjacent splice site, the reduction in Ri,total is quite significant and the difference in R_{i,total} values of the normal and mutant exon exceeds 3 bits (8 fold abundance), supporting a high level of exon skipping. We noted that instant invention presents potential cryptic isoforms with R_{i,total} values exceeding that of the mutated exon. Because the hnRNP A1 mutation affects acceptor site recognition, it is unlikely that these isoforms will be present, especially in instances where the cryptic splice site is a donor, and the natural acceptor is shared between the constitutive and cryptic isoforms.

Even assuming that triplet periodicity of exon lengths is random, one-third of all exon skipping events would not alter the reading frame. Nonsense mutations are generally acknowledged as pathogenic, are frequently lethal, and certainly reduce fecundity. It is well known in the art that non-sense codons induce exon skipping, as an alternative to nonsense mediated decay (T. Casci, Molecular evolution: Dealing with nonsense, Nature Reviews Genetics 12, 805). However, the specific mechanisms by which this phenomenon occurs have only been the subject of speculation, with limited specific evidence or mechanism as proven explanations for the phenomenon. Natural selection has evolved this mechanism to skip this abundant nonsense codon, TGA. For those exon skipping events that preserve the reading frame, the skipping event may result in less severe phenotypes, depending on how the structure of the protein is deformed by the loss of a stretch of amino acids. The periodic behavior of the gap surprisal function for exon lengths that are multiples of three nucleotides, suggests selection favoring exons of length that preserve the open reading frame.

The creation of an exon hnRNP A1 site can induce skipping, but as previously mentioned it can also enhance splicing by acting as a proofreader for exon recognition due to its preferential proximity to splice acceptor sites. To predict and validate which variants both create an hnRNP A1 site and increase skipping of the exon it is found in, we carried out the following steps of:
1. Use a high-throughput information-theory based tool to scan all variants in a dataset and determine which strengthen or create hnRNP A1 sites.
2. Create an hnRNP A1 site gap surprisal function based on the distance of pre-existing hnRNP A1 sites and natural exon sites by performing a complete genomic sequence scan for natural donor, natural acceptor and hnRNP A1 sites and creating a script to determine the exonic distances between them
3. Use information theory-based exon definition to factor the effect distance of the hnRNP A1 site from the natural site has on splicing, where the strength increase of hnRNP A1 site is subtracted by the aforementioned pre-computed gap surprisal value based on the frequency of distances of natural splice sites and pre-existing hnRNP A1 sites. This method was described for other splicing regulatory proteins in Mucaki et al. (2013), but was reported before the hnRNP A1 model was developed.
4. Use the Veridical method to demonstrate that significant reduction in the total information content of the exon containing the novel or strengthened hnRNPA1 site due to a mutation results in increased exon skipping in the mRNA of the individual carrying the mutation, but no increase in control individuals without this variant sequence.

The first 3 steps have been previously described in U.S. Ser. No. 14/154,905, where is the fourth step comprises an element of the instant invention. Because these mutations do not involve mutations at the natural splice sites, it would not be obvious to one of skill in the art that they would cause exon skipping. With this step, we support the predicted mutations with experiment data and and corresponding algorithm, that specifically distinguishing the present art from U.S. patent Ser. No. 14/154,905.

Information theory-based exon definition calculations have many advantages that are not present when only considering the created hnRNP A1 site. It takes the distance of the hnRNP A1 site to the closest natural site into account, as ones close to the natural site are more likely to have a positive influence on exon retention. We have found that a moderate to strong site (>4 bits) situated at least 50 nt from a splice junction induces exon skipping. This is because the negative contribution of the gap surprisal term of the Ri,total calculation rises very quickly as the distance from the splice junction increases. Additionally, variants in the first few nucleotides of the exon could simultaneously affect the natural site and create an hnRNP A1 site. The information theory exon definition calculation is that it takes into account the impact of both simultaneously, and the change in Rᵢ,total will reflect this. Variants were segregated based on whether they were predicted to increase or decrease total exon information content (hnRNP A1 influences considered in calculation). Finally, it has the distinct advantage of being able to predict the splicing outcome quantitatively, as in predicting the degree of decreased wildtype exon inclusion (Mucaki et al., 2013).

All variants called from 447 tumour and 106 normal breast tissue exomes by DNA sequencing, and RNA-seq transcriptome data associated with these same tissues, were obtained from The Cancer Genome Atlas hosted by the US National Cancer Institute (TCGA). All single nucleotide variants (SNPs) were scanned with the previously mentioned hnRNP A1 model. Then, RNA-seq for the flagged variants in these tumour and normal breast tissue samples were then analyzed using the Veridical program. The data was then filtered for variants calculated to significantly increase skipping (p<0.05). Exon skipping reads in the RNAseq indicated by Veridical were confirmed by visually inspecting the reads using the Integrative Genome Viewer (IGV). TCGA variants found to create or strengthen an hnRNP A1 site, significantly increase exon skipping using Veridical.

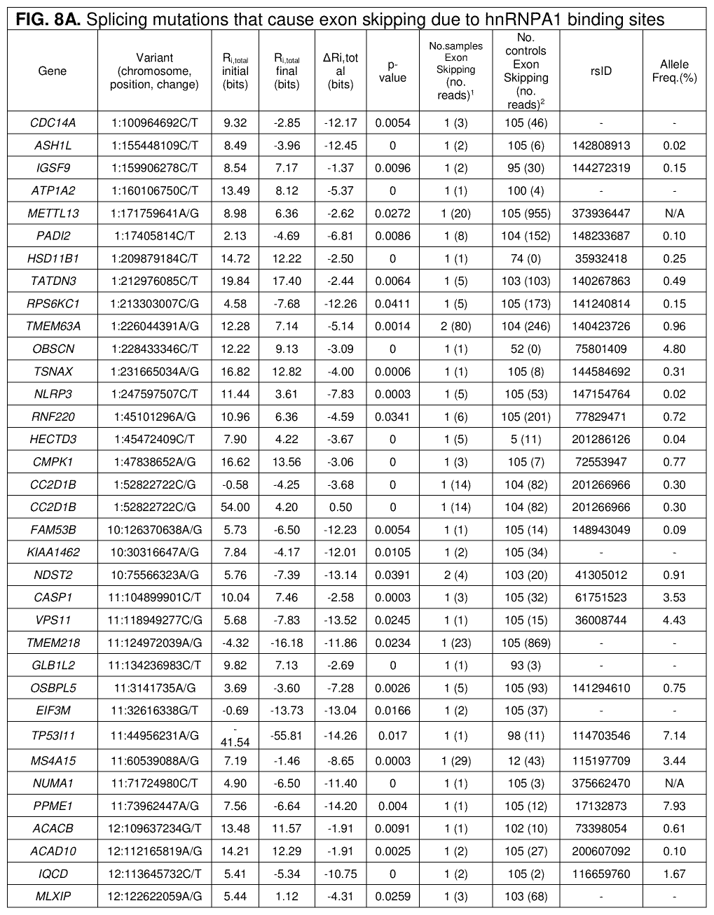

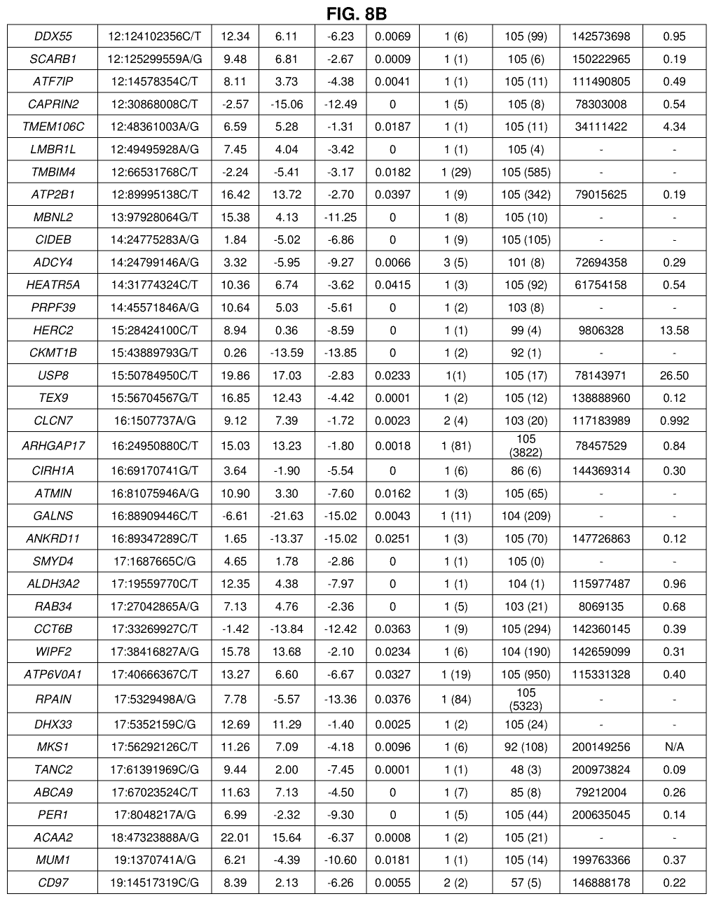

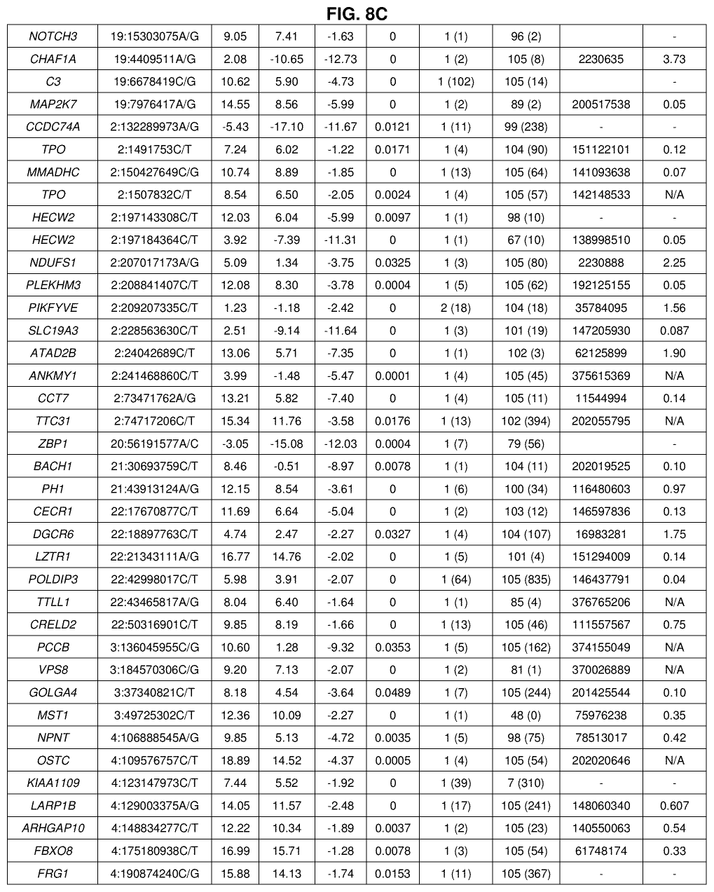

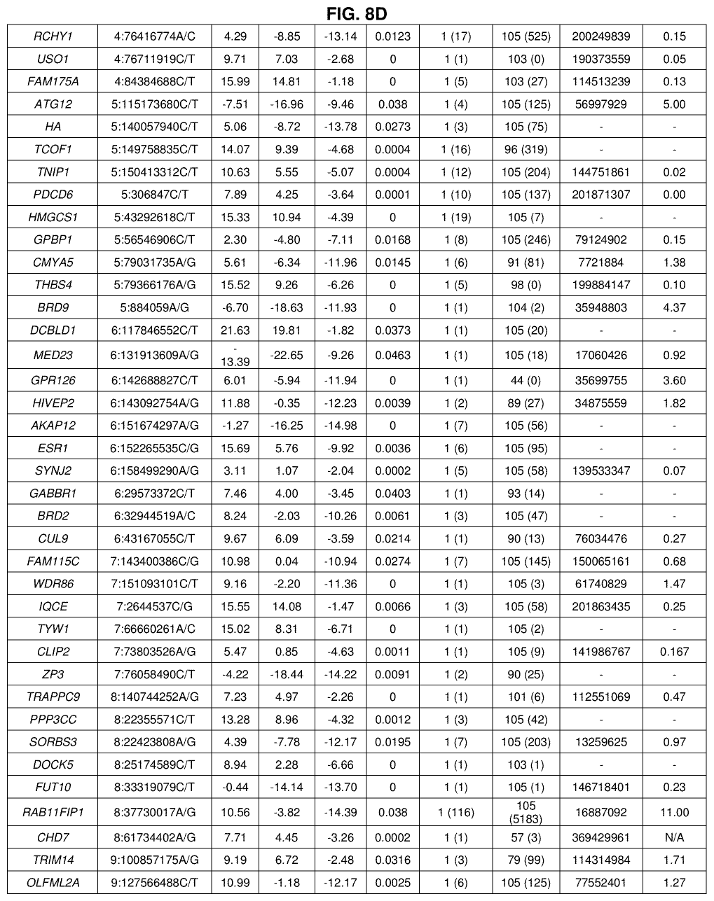

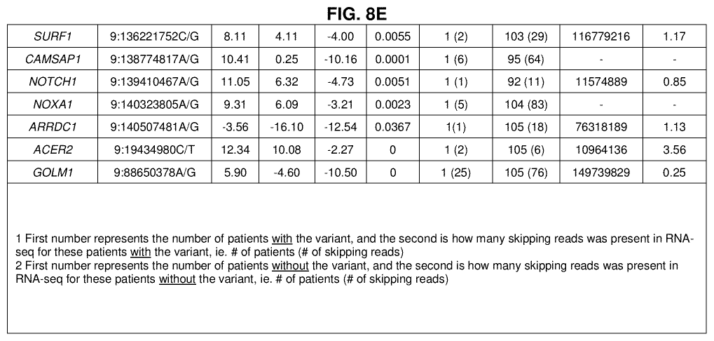

Exon skipping is found to be significant by Veridical far more often when R_{i,total} is decreasing. FIG. 8 contains a list of variants found in 106 normal breast tissue samples, where the Ri,total is predicted to decrease in total strength, and the p-value for variant-induced exon skipping is <0.05 for at least 1 patient (n=156). These variants are those most likely to have an effect on exon retention. By contrast 1054 total variants found to increase Ri,total, only 11 have exon skipping-related p-values less than 0.05. We highlight two representative examples from Table 1 to illustrate the effects of hnRNP A1 site activation on exon recognition. The SNP rs35784095 (allele frequency 1.56%) is a synonymous variant but creates a 4.7 bit hnRNP A1 site in exon 32 of the gene, decreasing the Ri,total by 2.4 bits. codes for a kinase that regulates endomembrane homeostasis, and mutations in this gene have been shown to cause corneal fleck dystrophy (CFD). This variant was called in 2 of the 106 TCGA normals, and Veridical indicated both individuals in having highly significant exon skipping (p-value<0.0001). Skipping of this exon would maintain the reading frame, which may explain why variant is found in the normal population. Veridical counted a combined 18 skipping reads between the two individuals, and another 18 skipping reads between the remaining 104 TCGA normals RNAseq data files, which suggests this variant is modulating skipping. The variant also creates a cryptic donor 40nt away from the natural donor (of equivalent strength), but there is no evidence of used in the RNAseq data. Similarly, rs117183989 is an uncommon SNP (allele frequency 0.992%) which is found in exon 8 of, and found to create a 2.5 bit hnRNP A1 site (ΔRi,total decrease of 1.7 bits) and was flagged by Veridical to induce exon skipping in the 2 TCGA normal samples with the variant. Defects in the gene, which encodes for a chloride channel transporter protein, can cause osteoporosis autosomal recessive type 4 (OPTB4) and autosomal dominant osteoporosis type 2 (OPTA2), but the variant may not trigger these conditions as the gene would retain its reading frame if the exon were skipped.

To illustrate the impact this exon skipping mechanism could have on human disease, all single-nucleotide variants (SNPs) from ClinVar, a database of human variants and their resulting phenotypes (with evidence), were downloaded, scanned with the hnRNP A1 model, and their effect on exon definition (Ri,total) was calculated. Of the 1484 nonsense, 3660 missense and 842 synonymous variants from ClinVar found to create an hnRNP A1 site, approximately 75% were found to decrease Ri,total when taking the hnRNP A1 site into account (1115 nonsense, 2723 synonymous and 636 synonymous variants; 75.1%, 74.4% and 75.5%, respectively). Nearly half of the nonsense, missense and synonymous variants are creating what are considered strong Ri,total decreases of 6 bits or more (45.6%, 47.3% and 50.0%, respectively). This is illustrated in more detail in FIG. 4. There does not seem to be a bias towards any specific type of mutation. However, variants which caused nonsense mutations were further investigated as exon skipping could be advantageous in these cases. A histogram of the relative frequency of ΔRi,total changes for all types of nonsense mutations are found in FIG. 5. Another term for a nonsense mutation is an amber mutation. Arginine>amber (CGA->TGA), Glutamate>amber (GAA->TAA or GAG->TAG), and Glycine>amber (GGA->TGA) mutations seem to have a greater percentage of variants decreasing Ri,total (78.5%, 81.0% and 84.2% reducing Ri,total, respectively). Cysteine>amber and Leucine->amber mutations are less likely to decrease Ri,total (46.3% and 43.8% variants reduce Ri,total, respectively), however these are predominantly due to the preponderance of specific nonsense nucleotide changes (AGA->TGA for Cysteine, AAA->TAA and AAG->TAG for Leucine; FIG. 6). Cysteine->amber mutation TGC->TGA and the Leucine->amber mutation GGA->TGA can lead to both increases or decreases to the total information content of the exon R_{i,total}. hnRNPA1 mutations with negative ARi,total values is proven that result in exon skipping occur in the exon that is skipped, typically occur in Arginine>amber, Glutamine>amber, Glutamate>amber, Glycine>amber, Serine>amber, Tryptophan>amber and Tyrosine>amber coding mutations, however certain missense and synonymous changes may less frequently create or strengthen hnRNPA1 sites. The frequency of reads in mRNA supporting exon skipping must be significantly greater than controls (resulting in p values that exclude the null hypothesis of normal splicing of <0.0001, or even <0.01, in most instances <0.05). The published literature (including Mucaki et al., 2013) does not anticipate this finding. By using information theory and Veridical, 193 variants predicted to create an hnRNP A1 site, lower the strength of the exon (Rᵢ,total), have significantly increased exon skipping reads in RNAseq data with these variants. Variants which both increased Rᵢ,total and skipping reads are extremely uncommon. and represent between 1-2% of the total variants tested by Veridical. Nonetheless, the method developed has the ability to find and support instances where created hnRNP A1 sites have an effect on splicing.

Changes may be made in the above methods without departing from the scope hereof. It should be noted that the matter contained in the above description or shown in the accompanying drawings should be interpreted as illustrative and not in a limiting sense. The following claims are intended to cover generic and specific features described herein, as well as statements of the scope of the present methodology, which, as a matter of language, might be said to fall therebetween.

It should be understood that suitable equivalents may be used in place of or in addition to the various instruments, components or compositions, the function and use of such substitute or additional components being held to be familiar to those skilled in the art and are therefore regarded as falling within the scope of the present disclosure. Therefore, the present examples are to be considered as illustrative and not restrictive, and the present disclosure is not to be limited to the details given herein but may be modified within the scope of the appended claims.

### REFERENCES

The following references are either cited in this disclosure or are of relevance to the present disclosure. All documents listed below, along with other papers, patents and publication of patent applications cited throughout this disclosures, are hereby incorporated by reference as if the full contents are reproduced herein.
Barash, Y., Calarco, J. A., Gao, W., Pan, Q., Wang, X., Shai, O., Blencowe, B. J., Frey, B. J. 2010. Deciphering the splicing code. Nature 465(7294): 53-9, 2010.
Berget S M. 1995. Exon recognition in vertebrate splicing. J Biol Chem. 270:2411-2414.
Bolisetty M T, Beemon K L. 2012. Splicing of internal large exons is defined by novel cis-acting sequence elements. Nucleic Acids Res. 40(18):9244-54.
Cartegni L., Krainer A. R. 2002. Disruption of an SF2/ASF-dependent exonic splicing enhancer in SMN2 causes spinal muscular atrophy in the absence of SMN1. Nat. Genet. 30:377-384.
Churbanov A, Igor B. Rogozin, Jitender S. Deogun and Hesham Ali, Method of predicting Splice Sites based on signal interactions, Biology Direct 1(2006), no.10.
Churbanov A, Igor Vorechovsky and Chindo Hicks A method of predicting changes in human gene splicing induced by genetic variants in context of cis-acting elements, BMC Bioinformatics 2010, 11:22
Claes K, Vandesompele J, Poppe B, Dahan K, Coene I, De Paepe A, Messiaen L. 2002. Pathological splice mutations outside the invariant A G/G T splice sites of BRCA1 exon 5 increase alternative transcript levels in the 5′ end of the BRCA1 gene. Oncogene. 21:4171-4175.
Claes K, Poppe B, Machackova E, Coene I, Foretova L, De Paepe A, and Messiaen L. 2003. Differentiating pathogenic mutations from polymorphic alterations in the splice sites of BRCA1 and BRCA2. Genes Chromosomes Cancer. 37:314-320.
Clark F, Thanaraj T A. 2002. Categorization and characterization of transcript-confirmed constitutively and alternatively spliced introns and exons from human. Hum Mol Genet. 11: 451-464.
Clavero S, Pérez B, Rincón A, Ugarte M, Desviat L R. 2004. Qualitative and quantitative analysis of the effect of splicing mutations in propionic acidemia underlying non-severe phenotypes. Hum Genet. 115(3):239-47.
Cook K B, Kazan H, Zuberi K, Morris Q, and Hughes T R. 2011. RBPDB: a database of RNA-binding specificities. Nucleic Acids Res. 39:D301-8.
Cover T M, Thomas J A. 2006. Elements of information theory. Wiley-Interscience, Hoboken, N.J.: p.748.
Dalgleish R, Flicek P, Cunningham F, Astashyn A, Tully R E, Proctor G, Chen Y, McLaren W M, Larsson P, Vaughan B W, Beroud C, Dobson G et al. 2010. Locus Reference Genomic sequences: an improved basis for describing human DNA variants. Genome Med. 2:24.
De Conti L, Baralle M, Buratti E. 2012. Exon and intron definition in pre-mRNA splicing. Wiley Interdiscip Rev RNA. doi: 10.1002/wrna.1140.
Divina P, Kvitkovicova A, Buratti E, Vorechovsky I. 2009. Ab initio prediction of mutation-induced cryptic splice-site activation and exon skipping. Eur J Hum Genet. 17:759-765.
Dominski Z, Kole R. 1991. Selection of splice sites in pre-mRNAs with short internal exons. Mol Cell Biol. 11(12):6075-83.
Dominski Z, Kole R. 1992. Cooperation of pre-mRNA sequence elements in splice site selection. Mol Cell Biol. 12:2108-2114.
Goina E, Skoko N, Pagani F. 2008. Binding of DAZAP1 and hnRNPA1/A2 to an exonic splicing silencer in a natural BRCA1 exon 18 mutant. Mol Cell Biol. 28(11):3850-60.
Graveley B R, Maniatis T. 1998. Arginine/serine-rich domains of S R proteins can function as activators of pre-mRNA splicing. Mol Cell. 1:765-771.
Goren A, Kim E, Amit M, Vaknin K, Kfir N, Ram O, Ast G. 2010. Overlapping splicing regulatory motifs—combinatorial effects on splicing. Nucleic Acids Res. 38:3318-3327.
Hwang D Y, Cohen J B. 1997. U1 small nuclear RNA-promoted exon selection requires a minimal distance between the position of U1 binding and the 3′ splice site across the exon. Mol Cell Biol. 17:7099-7107.
Ibrahim E C, Schaal T D, Hertel K J, Reed R, Maniatis T. 2005. Serine/arginine-rich protein-dependent suppression of exon skipping by exonic splicing enhancers. Proc Natl Acad Sci USA. 102:5002-5007.
Jaynes E. Information Theory and Statistical Mechanics. Phys. Rev. 106, 620-630 (1957).
Lim K H, Ferraris L, Filloux M E, Raphael B J, Fairbrother W G. 2011. Using positional distribution to identify splicing elements and predict pre-mRNA processing defects in human genes. Proc Natl Acad Sci USA. 108(27):11093-8.
Liu H X, Zhang M, Krainer A R. 1998. Identification of functional exonic splicing enhancer motifs recognized by individual S R proteins. Genes Dev. 12:1998-2012.
Liu H X, Chew S L, Cartegni L, Zhang M Q, Krainer A R. 2000. Exonic splicing enhancer motif recognized by human SC35 under splicing conditions. Mol. Cell. Biol. 20:1063-1071.
Macias-Vidal J, Rodes M, Hernandez-Perez J M, Vilaseca M A, Coll M J. 2009. Analysis of the CTNS gene in 32 cystinosis patients from Spain. Clin Genet. 76:486-489.
Mucaki E J, Ainsworth P, Rogan P K. 2011. Comprehensive prediction of mRNA splicing effects of BRCA1 and BRCA2 variants. Hum Mutat. 32:735-42.
Nalla V K, Rogan P K. 2005. Automated splicing mutation analysis by information theory. Hum Mutat. 25:334-342.
Robberson B L, Cote G J, and Berget S M. 1990. Exon definition may facilitate splice site selection in RNAs with multiple exons. Mol Cell Biol. 10:84-94.
Rogan P K, Faux B M, Schneider T D. 1998. Information analysis of human splice site mutations. Hum Mutat. 12:153-171.
Rogan P K, Svojanovsky S R, Leeder J S. 2003. Information theory-based analysis of CYP219, CYP2D6 and CYP3A5 splicing mutations. Pharmacogenetics. 13:207-18.
Rogan P K. 2009. Ab Initio Exon Definition Using an Information Theory-based Approach. Biochemistry Publications. Paper 10. http://ir.lib.uwo.ca/biochempub/10.
Rutter J L, Goldstein A M, Davila M R, Tucker M A, Struewing J P. 2003. CDKN2A point mutations D153sp1(c.457G>T) and IVS2+1G>T result in aberrant splice products affecting both p16INK4a and p14ARF. Oncogene. 22:4444-8.
Sanz D J, Acedo A, Infante M, Duran M, Perez-Cabornero L, Esteban-Cardenosa E, Lastra E, Pagani F, Miner C, Velasco E A. 2010. A high proportion of DNA variants of BRCA1 and BRCA2 is associated with aberrant splicing in breast/ovarian cancer patients. Clin Cancer Res. 16:1957-67.
Schneider T D, Stormo G D, Yarus M A, Gold L. 1984. Delila system tools. Nucleic Acids Res. 12:129-140.
Schneider T D. 1997. Information content of individual genetic sequences. J Theor Biol. 189:427-441.
Shultzaberger R K, Bucheimer R E, Rudd K E, Schneider T D. 2001. Anatomy of Escherichia coli ribosome binding sites. J Mol Biol. 313:215-228.
Smith P J, Zhang C, Wang J, Chew S L, Zhang M Q, Krainer A R. 2006. An increased specificity score matrix for the prediction of SF2/ASF-specific exonic splicing enhancers. Hum Mol Genet. 15(16):2490-508.
Spurdle A B, Healey S, Devereau A, Hogervorst F B, Monteiro A N, Nathanson K L, et al. ENIGMA—evidence-based network for the interpretation of germline mutant alleles: an international initiative to evaluate risk and clinical significance associated with sequence variation in BRCA1 and BRCA2 genes. Hum Mutat. 2012:33(1)2-7.
Stamm S, Riethoven J J, Le Texier V, Gopalakrishnan C, Kumanduri V, Tang Y, Barbosa-Morais N L, Thanaraj T A. 2006. ASD: a bioinformatics resource on alternative splicing. Nucl Acids Res. 34(suppl 1):D46-55.
Thomassen M, Ana Blanco, Marco Montagna, Thomas V. O. Hansen, Inge S. Pedersen, Sara Gutierrez-Enriquez, Mireia Menendez, Laura Fachal, Marta Santamarina, Ane Y. Steffensen, Lars Jonson, Simona Agata, Phillip Whiley, Silvia Tognazzo, Eva Tornero, Uffe B. Jensen, Judith Balmana, Torben A. Kruse, David E. Goldgar, Conxi Lazaro, Orland Diez, Amanda B. Spurdle, Ana Vega, Characterization of BRCA1 and BRCA2 splicing variants: a collaborative report by ENIGMA consortium members Breast Cancer Res Treat. 2012 April; 132(3):1009-23
Tompson S W, Ruiz-Perez V L, Blair H J, Barton S, Navarro V, Robson J L, Wright M J, Goodship J A. 2007. Sequencing EVC and EVC2 identifies mutations in two-thirds of Ellis-van Creveld syndrome patients. Hum Genet. 120:663-670.
Tribus M. 1961. Thermostatics and thermodynamics: an introduction to energy, information and states of matter, with engineering applications. Van Nostrand, Princeton, N.J.: p. 649.

1. A method of diagnosing one or more mutations in an individual that are associated with either an inherited genetic disease, predisposition to genetic disease, or with cancer and caused by mRNA splicing defects, by detecting and validating abnormal splicing in a transcriptome of said individual by computer-implemented, high throughput sequence analysis, said method comprising:
a) predicting mutations in said individual DNA sequence data showing a predicted mutant allele documented by multiple sequence reads, based upon first determining their gene or genome sequences, that alter the spliced structures of one or more mRNA transcripts;
b) extracting and reverse transcribing mRNA into cDNA from said individual, and characterizing the isoforms of each expressed, mutated gene by:
(i) for each predicted mutation in step a) that occurs within the nucleotide sequences that either define a natural splice junction of an exon or occur within intronic sequences adjacent to a natural splice junction, determining the sequence of and counting the number of sequenced cDNA templates in a sequence library containing at least one intronic nucleotide in a sample, ζᵢ, as the evidence for intron inclusion in said individual;
(ii) for each predicted mutation in step a) that occurs within the nucleotide sequences that either define a natural splice junction of an exon or occur within intronic sequences adjacent to a natural splice junction, determining the sequence of and counting ζᵢ, as the evidence for intron inclusion in non-disease samples, from the number of sequence reads derived from cDNA templates containing at least one intronic nucleotide identified in step b)(i) in said individual and in one or more non-disease samples, a population of non-disease samples demonstrating the same predicted splicing mutation present in said individual occurring in less than 1% of their corresponding genomic sequences; and
(iii) for each predicted mutation in step a), determining the probability that the mutation alters the mRNA structure of a gene from the count of sequence reads in the sample containing the predicted mutation determined in step b)(i) and the number of counts of sequence reads in the set of N−1 non-diseased samples computed in step b)(ii), as:
$$μ = \frac{{\sum }_{j = 1}^{N} {V}_{j}}{N} σ = \sqrt{\frac{1}{N} {\sum }_{j = 1}^{N} {( {V}_{j} - \overline{V} )}^{2}}$$
$$z = \frac{\vert {Ϛ}_{i} \vert - μ}{σ} p = Φ ( ψ ( z , \frac{1}{2} ) )$$
where
$$Φ ( ψ ( z , \frac{1}{2} ) )$$
represents the cumulative distribution function of read counts of the one-sided (right-tailed, i.e. P[X>x]) standard normal distribution with mean μ and standard deviation σ,
$$ψ ( z , \frac{1}{2} )$$
is the z score of the Yeo-Johnson transformation of ζᵢ read counts z is the distance from μ for ζᵢ reads counts, p is the probability determined for z, N represents the total number of samples and Vᵢ represents the set of all ζᵢ validations for each sample j, across all N samples, and V̅ is the average ζₑ read counts among the non-disease samples; and
c) validating that a predicted mutation results in mRNA isoforms with intron inclusion with structures containing ζᵢ, if the probability of sequence read evidence present in said individual from step b)(iii) is less than or equal to 0.05499.

2. The method of claim 1, further comprising graphically displaying ζᵢ for a validated mutation in said individual.

3. The method of claim 1, where the counts of the sequence reads in all of the samples are transformed to a normal distribution prior to computing the probability.

4. The method of claim 3, in which the splicing mutation either inactivates a natural or constitutive splice site or activates an intronic cryptic splice site.

5. The method of claim 1, in which the splicing mutation either inactivates a natural or constitutive splice site or activates an intronic cryptic splice site.

6. A method of diagnosing one or more mutations in an individual that are associated with either an inherited genetic diseases, predisposition to genetic disease, or with cancer and caused by mRNA splicing defects, by detecting and validating abnormal splicing in a transcriptome of said individual by computer-implemented high throughput sequence analysis, said method comprising:
a) predicting mutations in said individual DNA sequence data showing a predicted mutant allele documented by multiple sequence reads, based upon first determining their gene or genome sequences, that alter the spliced structures of one or more mRNA transcripts;
b) extracting and reverse transcribing mRNA into cDNA from said individual, and characterizing the isoforms of each expressed, mutated gene by:
(i) for each predicted mutation in step a) that occurs within the nucleotide sequences that define a natural splice junction of an exon, determining the sequence of and counting the number of sequenced cDNA templates in a sequence library containing the abnormal splice junction derived from non-consecutive exons from the same gene in a sample, ζₑ, as the evidence for exon skipping in said individual;
(ii) for each predicted mutation in step a) that occurs within the nucleotide sequences that define a natural splice junction of an exon, determining the sequence of and counting the number of sequenced cDNA templates in a sequence library containing the same abnormal splice junction derived from the same gene in a sample, ζₑ, as the evidence for exon skipping in the non-disease samples, from the number of sequence reads derived from cDNA templates containing the same abnormal splice junction identified in step b)(i) in said individual and in one or more non-disease samples, a population of non-disease samples demonstrating-the same predicted splicing mutation present in said individual occurring in less than 1% of their corresponding genomic sequences; and
(iii) for each predicted mutation in step a), determining the probability, P, that the mutation alters the mRNA structure of a gene from the count of sequence reads in the sample containing the predicted mutation determined in step b)(i) and the number of counts of sequence reads in the set of N−1 non-disease samples computed in step (ii), as:
$$μ = \frac{{\sum }_{j = 1}^{N} {V}_{j}}{N} σ = \sqrt{\frac{1}{N} {\sum }_{j = 1}^{N} {( {V}_{j} - \overline{V} )}^{2}}$$
$$z = \frac{\vert {Ϛ}_{e} \vert - μ}{σ} p = Φ ( ψ ( z , \frac{1}{2} ) )$$
where
$$Φ ( ψ ( z , \frac{1}{2} ) )$$
represents the cumulative distribution function of read counts of the one-sided (right-tailed, i.e. P[X>x]) of the standard normal distribution with mean μ and standard deviation σ,
$$ψ ( z , \frac{1}{2} )$$
is the z score of the Yeo-Johnson transformation of ζₑ read counts, z is the distance from μ for ζₑ read counts (also known as the standard normal deviate), p is the probability determined for z, N is the total number of samples and Vᵢ represents the set of all ζₑ validations for each sample j, across all N samples, and V̅ is the average ζₑ read counts among the non-disease samples; and
c) validating that a predicted mutation results in mRNA isoforms with exon skipping with structures containing ζₑ, if the probability of sequence read evidence present in said individual from step b)(iii) is less than or equal to 0.05499.

7. The method of claim 6, further comprising graphically displaying ζₑ for a validated mutation in said individual.

8. The method of claim 6, where the counts of the sequence reads in all of the samples are transformed to a normal distribution prior to computing the probability.

9. The method of claim 8, in which the splicing mutation is leaky and has a partial effect, reducing the amount of normal mRNA splicing, thereby reducing the number of sequence reads corresponding to the constitutively spliced mRNA, such that the probability of observing a non-disease sample with this reduced read count is less than 0.05499.

10. The method of claim 6, in which the splicing mutation is leaky and has a partial effect, reducing the amount of normal mRNA splicing, thereby reducing the number of sequence reads corresponding to the constitutively spliced mRNA, such that the probability of observing a non-disease sample with this reduced read count is less than 0.05499.

11. The method of claim 6, in which the splicing mutation alters the information content an mRNA sequence bound by a factor that regulates normal mRNA splicing and causes exon skipping.

12. The method of claim 6, in which the splicing mutation alters the total exon information and causes exon skipping.

13. A method of diagnosing one or more mutations in an individual that are associated with either an inherited genetic diseases, predisposition to disease or with cancer and caused by mRNA splicing defects, by detecting and validating abnormal splicing in a transcriptome of said individual by computer-implemented high throughput sequence analysis, said method comprising:
a) predicting mutations in said individual DNA sequence data showing a predicted mutant allele documented by multiple sequence reads, based upon first determining their gene or genome sequences, that alter the spliced structures of one or more mRNA transcripts;
b) extracting and reverse transcribing mRNA from a cell from said individual, and characterizing the isoforms of each expressed, mutated gene by:
(i) for each predicted mutation present in the corresponding genomic sequence adjacent to a cryptic splice junction, determining the sequence of and counting the number of sequenced cDNA templates in a sequence library containing the same abnormal splice junction derived from the same gene in a sample, ζ_{c}, as the evidence for cryptic splicing in said individual;
(ii) for each predicted mutation in step a), counting ζ_{c}, as the evidence for cryptic splicing in the non-disease samples, from the number of sequence reads derived from cDNA templates containing the same cryptic splice site present in the sample previously found in said individual and in one or more non-disease samples, a population of non-disease samples demonstrating the same predicted splicing mutation present in said individual occurring in less than 1% of their corresponding genomic sequences; and
(iii) for each predicted mutation, determining the probability, P, that the mutation alters the mRNA structure of a gene from the count of sequence reads in the sample predicted to result from the mutation determined in step b)(i) and the number of counts of sequence reads derived by cryptic splicing in the set of N−1 non-disease samples computed in step b)(ii), as:
$$μ = \frac{{\sum }_{j = 1}^{N} {V}_{j}}{N} σ = \sqrt{\frac{1}{N} {\sum }_{j = 1}^{N} {( {V}_{j} - \overline{V} )}^{2}}$$
$$z = \frac{\vert {Ϛ}_{c} \vert - μ}{σ} p = Φ ( ψ ( z , \frac{1}{2} ) )$$
where
$$Φ ( ψ ( z , \frac{1}{2} ) )$$
represents the cumulative distribution function of read counts of the one-sided (right-tailed, i.e. P[X>x]) standard normal distribution with mean μ and standard deviation σ,
$$ψ ( z , \frac{1}{2} )$$
is the z score of the Yeo-Johnson transformation of ζ_{c} read counts, z is the distance from μ for ζ_{c} read counts, p is the probability determined for z, N is the total number of samples, Vᵢ represents the set of all ζ_{c} validations for each sample j across all N samples, and \hat{V} is the average ζ_{c} read counts among the non-disease samples; and
c) validating that a predicted mutation results in cryptic mRNA isoforms with structures containing ζ_{c}, if the probability of observing the sequence read evidence present in said individual from step b)(iii) is less than or equal to 0.05499.

14. The method of claim 13, further comprising graphically displaying ζ_{c} for a validated mutation in said individual.

15. The method of claim 13, where the counts of the sequence reads in all of the samples are transformed to a normal distribution prior to computing the probability.

16. The method of claim 15, in which the splicing mutation inactivates a constitutive splice site and activates a cryptic splice site.

17. The method of claim 13, in which the splicing mutation inactivates a constitutive splice site and activates a cryptic splice site.
